SEQUENCE LISTING (22 sequences)

SEQ ID NO: 1 - DNA, 20 bp, Artificial Sequence
  Oligonucleotide primer
cattgcttgcactgactatt

SEQ ID NO: 2 - DNA, 20 bp, Artificial Sequence
  Oligonucleotide primer
aggatcgactcggatgttcc

SEQ ID NO: 3 - DNA, 20 bp, Artificial Sequence
  Oligonucleotide primer
ggagtgccgttcagaatcgt

SEQ ID NO: 4 - DNA, 20 bp, Artificial Sequence
  Oligonucleotide primer
accatcaaatgaatatccat

SEQ ID NO: 5 - DNA, 27 bp, Artificial Sequence
  Oligonucleotide primer
gccggatccgcccagttgcccgacttt

SEQ ID NO: 6 - DNA, 28 bp, Artificial Sequence
  Oligonucleotide primer
ctgaaagcttttagaatttcacgccgac

SEQ ID NO: 7 - DNA, 20 bp, Artificial Sequence
  Oligonucleotide primer
cactgcttgcactgactatt

SEQ ID NO: 8 - DNA, 20 bp, Artificial Sequence
  Oligonucleotide primer
aggatcgactcggatgttcc

SEQ ID NO: 9 - DNA, 20 bp, Artificial Sequence
  Oligonucleotide primer
gacaatctatcgattgtatg

SEQ ID NO: 10 - DNA, 20 bp, Artificial Sequence
  Oligonucleotide primer
gtccaacatcaatacaacct

SEQ ID NO: 11 - DNA, 20 bp, Artificial Sequence
  Oligonucleotide primer
ttcgttacctccggcatccg

SEQ ID NO: 12 - DNA, 20 bp, Artificial Sequence
  Oligonucleotide primer
accatcaaatgaatatccat

SEQ ID NO: 13 - protein, 263 aa, Neisseria polysaccharea
MKKALLALTIAAISGTAMAQLPDFLGKGEYTVRTDISKQTLKNADLKEKHKVQKNIGFRADMPFDDIHGMRFEVSHSRDKKDMYDVTESTTTPYGKDIKEKRTDVYAGYTYTQPLSETTKLRAGLGLGYEKYKDMQINKKGTELNVEREAFYTKAHADLTSDLGSGWYLNPWAEVKVDLNAKLKQNSTVAGASADINAKTRGWGVGVGANIGKQITDTVGIEAGPFYKHRYFKASGNFVLDGENSRVDPTKINEYGVRVGVKF

SEQ ID NO: 14 - protein, 261 aa, Neisseria polysaccharea
MKKALLALTIAAISGTAMAQLPDFLGKGEYTIRTDIAKQTLKNADLKEKHKVQKNIGFRADMPFDDIHGMRFEVSHSRDKKDLYDVTESTTKPYGKDIKEKRTDVYAGYTYTQPLSETTKLRAGLGLGYEKYKDTQTGKATINDEREAFYTKAHADLTSDLGSGWYLNPWAEVKVDLNAKLKQNSTAAGASADINAKTRGWGVGVGANIGKQITDTVGIEAGPFYKHRHFKASGNFVLDGENTRVDPTKINEYGVRVGVKF

SEQ ID NO: 15 - protein, 263 aa, Neisseria gonorrhoeae
MKKALLALTIAAISGTAMAQLPDFLGKGEYTVRTDISKQTLKNADLKEKHKVQKNIGFRADMPFDDIHHGMRFEVSHSRDKKDMYVVTESTTKPFGKDVKEKRTDVYAGYTYTQPISEATKLRAGLGLGYEKYKDAVANEKGTVSTEREAFYTKAHADLTSDLGGGWYLNPWAEVKVDLDAKLKHNATVAGVSADINAKTRGWGVGVGANIGKQITDTVGIEAGPFYKHRHFKASGSFVLDGGNIRVDPTKINEYGVRVGVKF

SEQ ID NO: 16 - protein, 262 aa, Neisseria gonorrhoeae
MKKALLALTIAAISGTAMAQLPDFLGKGEYTVRTDISKQTLKNADLKEKHKVQKNIGFRADMPFDDIHHGMRFEVSHSRDKKDMYVVTESTTKPFGKDVKEKRTDVYAGYTYTQPISEATKLRAGLGLGYEKYKDAVANEKGTVSTEREAFYTKAHADLTSDLGGWYLNPWAEVKVDLDAKLKHNATVAGASADINAKTRGWGVGVGANIGKQITDTVGIEAGPFYKHRHFKASGSFVLDGGNIRVDPTKINEYGVRVGVKF

SEQ ID NO: 17 - protein, 272 aa, Neisseria meningitidis
  misc_feature (156)..(156): Xaa can be any naturally occurring amino acid
MKKTVFTYAMIALTGTAAAAQELQIANEFTVHTDLSSISSTRAFLKEKHKAAKHISVRADILFDASQGIRLEAGFGRSKKNIINLETDENKLGKTKDVKLPTGVPENRIDFYTGYTYTQTLSDSLNFRVGAGLGFESSKDSIKTTKHTLHSSRQSXSAKVHADLLSQLGNGWYINPWSEVKFDKNSRYKLNTGVTSLKKDINQKTNGWGFGLGANIGKKLGESASIEAGPFYKQRTYKESDEFSVPTTSGDVSLTIPKTSIREYGLRVGIKF

SEQ ID NO: 18 - protein, 272 aa, Neisseria meningitidis
MKKTVFTCAMIALTGTAAAAQELQTANEFTVHTDLSSISSTRAFLKEKHKAAKHISVRADIPFDASQGIRLEAGFGRSKKNIINLETDENKLGKTKDVKLPTGVPENRIDLYTGYTYTQTLSDSLNFRVGAGLGFESSKDSIKTTKHTLHSSRQSWLAKVHADLLSQLGNGWYINPWSEVKFDLNSRYKLNTGVTNLKKDINQKTNGWGFGLGANIGKKLGESASIEAGPFYKQRTYKESGEFSVTTKSGDVSLTIPKTSIREYGLRVGIKF

SEQ ID NO: 19 - protein, 272 aa, Neisseria meningitidis
MKKTVFTCAMIALTGTAAAAQELQTANEFTVHTDLSSISSTRAFLKEKHKAAKHIGVRADIPFDANQGIRLEAGFGRSKKNIINLETDENKLGKTKNVKLPTGVPENRIDLYTGYTYTQTLSDSLNFRVGAGLGFESSKDSIKTTKHTLHSSRQSWLAKVHADLLSQLGNGWYINPWSEVKFDLNSRYKLNTGVTNLKKDINQKTNGWGFGLGANIGKKLGESASIEAGPFYKQRTYKESGEFSVTTKSGDVSLTIPKTSIREYGLRVGIKF

SEQ ID NO: 20 - DNA, 792 bp, Neisseria gonorrhoeae
atgaaaaaagcactgcttgcactgactattgccgccatctccggtactgctatggcccagttgcccgactttctgggtaaaggcgaatataccgtccgtacagacatctccaaacaaacgctgaaaaatgccgatttgaaagaaaaacacaaagtacaaaaaaacatcggtttccgtgccgatatgccgtttgacgatattcaccacggcatgcgtttcgaagtatcacacagccgagacaaaaaagacatgtacgttgtgaccgaaagtactactaaaccattcggcaaagacgttaaagaaaaacgcaccgacgtgtatgccggttacacctacactcaaccgatcagtgaagccaccaaactgcgtgccggtttaggtttgggctatgaaaaatacaaagatgcggtagctaatgagaaaggaacagtcagcaccgagcgcgaagccttctataccaaagcgcacgccgacctgacttccgatttgggcggcggctggtatctgaatccttgggcagaggttaaggttgacctggacgccaaattgaaacacaacgcaactgtagccggtgtctcagccgacattaacgcaaaaacacgcggatggggcgtgggcgtaggtgcgaatatcggcaaacaaattaccgataccgtcggcatcgaagccggcccgttctacaaacaccgtcacttcaaagcatccggcagcttcgttttagacggcgggaacatccgagtcgatcctaccaaaatcaacgaatacggtgttcgtgtcggcgtgaaattctaa

SEQ ID NO: 21 - protein, 263 aa, Neisseria gonorrhoeae; identical to SEQ ID NO: 15

SEQ ID NO: 22 - protein, 281 aa, Artificial Sequence
  Recombinant gonococcal OpcA protein used for mouse imminication
MRGSHHHHHHGMASMTGGQQMGRDLYDDDDKDRWGSAQLPDFLGKGEYTVRTDISKQTLKNADLKEKHKVQKNIGFRADMPFDDIHHGMRFEVSHSRDKKDMYVVTESTTKPFGKDVKEKRTDVYAGYTYTQPISEATKLRAGLGLGYEKYKDAVANEKGTVSTEREAFYTKAHADLTSDLGGGWYLNPWAEVKVDLDAKLKHNATVAGVSADINAKTRGWGVGVGANIGKQITDTVGIEAGPFYKHRHFKASGSFVLDGGNIRVDPTKINEYGVRVGVKF

(19) United States
(12) Patent Application Publication
(10) Pub. No.: US 2011/0182981 A1
(43) Pub. Date: Jul. 28, 2011

(54) GONOCOCCAL VACCINES

(76) Inventor: Peixuan Zhu, Potomac, MD (US)

(21) Appl. No.: 13/060,309

(22) PCT Filed: Aug. 24, 2009
(86) PCT No.: PCT/US09/54749

§ 371 Date: Mar. 28, 2011

Related U.S. Application Data
(60) Provisional application No. 61/091,503, filed on Aug. 25, 2008.

(51) Int. Cl. A61K 9/127; C07K 14/22; A61K 39/095; A61K 39/116; G01N 33/68; A61P 31/04; A61P 37/04

(52) U.S. Cl. 424/450; 530/350; 424/249.1; 424/203.1; 424/197.11; 436/501

(57) ABSTRACT

The present invention relates to immunogenic compositions comprising a recombinant Neisseria gonorrhoeae OpcA and methods of eliciting an immune response in a mammal by administering a formulation comprising N. gonorrhoeae OpcA or a portion or fragment of N. gonorrhoeae OpcA. The invention also provides for methods and kits for diagnosing N. gonorrhoeae infection using said recombinant N. gonorrhoeae OpcA.

### CROSS REFERENCE TO RELATED APPLICATIONS

This application claims the priority benefit of U.S. Provisional Patent Application Ser. No. 61/091,503, filed Aug. 25, 2008, incorporated herein by reference.

### GOVERNMENT SUPPORT

This invention was made with support from the government under SBIR Grant No. R43AI58331 awarded by the National Institutes of Health. The government has certain rights in the invention.

### FIELD OF THE INVENTION

The present invention related to recombinant technology, molecular immunology, and microbiology. More specifically, the present invention provides for an antigenic and immunogenic OpcA protein from Neisseria gonorrhoeae.

### BACKGROUND

Neisseria gonorrhoeae causes the sexually transmitted disease gonorrhoea, one of the most common sexually transmitted diseases worldwide. In the United States, gonorrhoea is second only to Chlamydia infections in the number of cases reported to the Centers for Disease Control and Prevention (CDC). The CDC estimates that each year more than 700,000 people in the United States will get a new gonorrheal infection. If left untreated, the bacteria can spread into the reproductive tract or, in rare cases, into the bloodstream and infect the joints, heart valves, or brain.

Although gonorrhea is still treatable with antibiotic therapy, the incidence of antibiotic resistance in N. gonorrhoeae is steadily increasing. For example, 31.6% of isolates collected in the United States in 1995 by the Gonococcal Isolate Surveillance Project were resistant to penicillin and/or tetracycline. Recently, resistance to the fluoroquinolones (ciprofloxacin and ofloxacin) has emerged. Resistance to CDC-recommended doses of ciprofloxacin and ofloxacin exceeds 10% in Hong Kong and Japan; more than 60% of isolates in some parts of the Republic of the Philippines are resistant to ciprofloxacin and ofloxacin. Currently, broad-spectrum cephalosporins are the only antimicrobial agents to which N. gonorrhoeae has not developed resistance.

Moreover, gonococcal infections, particularly in women, may ascend to more serious disease, including endometritis (infection of the uterine wall), salpingitis (infection of the fallopian tubes), ectopic pregnancy, and pelvic inflammatory disease (PID). The consequences of these advanced stages of gonococcal infection are quite severe, with infertility common and death a possibility if untreated. Over one million cases of PID occur every year, to which N. gonorrhoeae contributes up to 50% in endemic areas. The cost of treatment for PID was expected to reach $10 billion in the year 2000. In addition to the adverse consequences for reproductive health, infection with N. gonorrhoeae also facilitates both the acquisition and transmission of the human immunodeficiency virus.

Currently, there are no vaccines available for prevention of gonococcal disease, in part because N. gonorrhoeae lacks the cps locus on the bacterial genome and therefore does not express a capsule polysaccharide. Although several components on the bacterial cell surface, such as Pili, Tbp, PorB and LOS, have been investigated as the candidates of gonococcal vaccine, their immunogenic potential is limited by sequence and antigenic variability. Thus, although they induce protective antibodies against the homologous strain, these antigens fail to induce protection against heterologous strains. With few of exceptions, the surface-exposed proteins described during the past three decades suffer the drawback of this antigenic variability. Hence, the development of an effective vaccine against N. gonorrhoeae remains an important goal in achieving global prevention and control of this disease.

### SUMMARY OF THE INVENTION

An object of the present invention provides for an immunogenic composition against N. gonorrhoeae. One embodiment of the present invention provides for a gonococci surface antigen, the OpcA protein, which exhibits little variability amongst gonococci strains. In another embodiment of the invention, the OpcA protein is a recombinant protein, or a fragment or portion thereof. In a particular embodiment, the recombinant OpcA is expressed in and isolated from E. coli.

Another embodiment provides for a method of eliciting an immune response in animals and/or humans by administering at least one dose of a composition comprising N. gonorrhoeae OpcA or a fragment or portion thereof, with or without an adjuvant.

### DESCRIPTION OF THE DRAWINGS

FIG. 1 depicts the three-dimensional structure of N. meningitidis OpcA protein. Prince et al., 99 Proc. Nat'l Acad. Sci. U.S.A. 3417-21 (2002). Three Zn²⁺ ions are shown as spheres, and the two rings of hydrophobic residues are shown in light gray.

FIG. 2 illustrates five types of genetic organization at the opcA locus in Neisseria (Types I-V). Neisseria species are indicated as follows: Nm, N. meningitidis; Np, N. polysaccharea; Ng, N. gonorrhoeae. Strain names are in parentheses. Arrows indicate the open reading frames and small arrows indicate the primers used for PCR amplification. Zhu et al., 33 Mol. Microbiol. 635-50 (1999). The shadowed boxes with “C” indicate Correia element of 107 bp Toleman et al., 3 Cell. Microbiol. 33-44 (2001); 89 Zhu et al., 34 FEMS Immunol. Med. Microbiol. 193-200 (2002)), and small diamond with line indicates a frameshift mutation at orfY in strain FA1090. Dotted lines indicate deletions and the small triangles indicate direct repeats of nine nucleotides ACAGACATG (DR1 and DR2). The nucleotide sequences for the strains are under GenBank accession numbers: Z2491, AJ242841; MC58, AE002456; FA1090, AJ242839; 85322, AY072808; 89357, AY072809; FAM18, AJ242842; and 85321, AY072807.

FIG. 3 presents a modified multiple alignment of the deduced peptide sequence of OpcA from N. meningitidis (Nm) (SEQ ID NOs: 17-19), N. gonorrhoeae (Ng) (SEQ ID NOs:15-16) and N. polysaccharea (Np) (SEQ ID NOs: 13-14). The species and strain names are on the left. Thirteen Nm-OpcA sequence variants (Taha et al., 28 Mol Microbiol 1153-63 (1998)), is indicated with “Nm 13V”. The numbers above indicate positions. The numbers on the right indicate the length of OpcA peptide sequence. The Nm-OpcA sequence from strain Z2491 (3D crystal structure available) (Prince et al., 99 Proc. Natl. Acad. Sci. USA, 3417-21 (2002); Riou et al., 37 Intl J. Syst. Bacteriol. 163-65 (1987)), is on the bottom. The leader sequence and surface-exposed loops (loops 1 to 5) included. β transmembrane strands are underlined and periplasmic turns are shown in bold (Ta-Td) (Merker et al., 16 Annu. Rev. Cell Dev. Biol. 423-57 (2000); Prince et al., 2002; Riou et al., 37 Int'l J. Syst. Bacteriol. 163-65 (1987). β helix is shown in italics. Identity with the OpcA sequence of Nm strain Z2491 is indicated by a dash (-) and deletion by a period (.). Thirty-two polymorphic sites between Ng-OpcA and Np-OpcA are shadowed and there are seventeen specific polymorphic site differences among the three species.

FIG. 4 shows SDS-PAGE analysis for expression and purification of gonococcal OpcA protein. Lane 1: PageRuler prestained protein ladder (Fermentas Life Sciences); Lane 2: Expression control of pRSET/lacZ with the lacZ gene encoding for β-galactosidase that appears as a 120 KDa band on the gel; Lane 3, E. coli cell lysate before IPTG induction; Lane 4, E. coli cell lysate after IPTG induction; Lane 5, recombinant OpcA protein purified by affinity chromatography.

FIG. 5 depicts the construction and confirmation of the opcA isogenic mutant. Panel A presents a schematic presentation of the genetic map at the opcA locus in strain FA1090 and insertion site of kanamycin cassette (Kan). Panel B shows electrophoresis of restriction enzyme-digested plasmids. Lane 1, Sph I-digested plasmid pOPC-R11; Lane 2, Hinc II-digested plasmid pUC4Kan; Lane 3, BamH I-digested plasmid pOPC-RK13. (C)PCR confirmation of the opcA isogenic mutant. By using opcA-specific primers P299 (O15)/P300 (O16), an amplicon of 738 bp was obtained from FA1090 wild type strain (Lane 1), whereas larger amplicon of 1990 bp was obtained from the mutant strain FA1090ΔopcA (Lane 2), indicating that the Kan cassette has been integrated into the opcA gene. This has been further confirmed by using Kan-specific primers K1/K2, a specific amplicon of 542 bp was observed from the mutant strain FA1090ΔopcA (Lane 4) but not from the wild type strain (Lane 3).

FIG. 6 reflects the Southern blotting analysis of isogenic mutant FA1090ΔopcA and wild-type strain FA1090. Panel A: Hind III-digested genomic DNAs were probed by the opcA probe. Lane 1, mutant FA1090ΔopcA, Lane 2, wild type strain FA1090. Panel B: Hind III digested DNAs were probed by the Kan probe.

FIGS. 7A and 7B present the DNA and amino acids sequences, respectively, of the gonococcal OpcA protein from strain FA1090 (SEQ ID NOs: 20-21).

FIG. 8 shows the amino acid sequence of recombinant gonococcal OpcA protein (SEQ ID NO: 22) used for the mouse immunizations. Underlining indicates the sequence tag from the expression vector.

### DETAILED DESCRIPTION OF THE INVENTION

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims. Unless defined otherwise, all technical and scientific terms used herein have the same meaning as those commonly understood to one of ordinary skill in the art to which this invention pertains.

As used herein and in the claims, the singular forms “a,” “an,” and “the” include the plural reference and equivalents known to those skilled in the art unless the context clearly indicates otherwise. Other than in the operating examples, or where otherwise indicated, all numbers expressing quantities of ingredients or reaction conditions used herein should be understood as modified in all instances by the terms “about” or “approximately.”

All patents and other publications identified are incorporated herein by reference for the purpose of describing and disclosing, for example, the methodologies described in such publications that might be used in connection with the present invention, but are not to provide definitions of terms inconsistent with those presented herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on information available to the applicants and do not constitute any admission as to the correctness of the dates or contents of these documents.

The present invention provides for an isolated antigenic and immunogenic OpcA protein from N. gonorrhoeae. In one embodiment, the OpcA is recombinant. The present invention also provides for a method of eliciting an immunogenic response in a mammal by administering a formulation comprising N. gonorrhoeae OpcA. Another embodiment of the present invention provides for an immunogenic gonococcal OpcA protein that raises protective immunity. Yet another embodiment provides for the biological function of gonococcal opcA gene.

In the U.S. alone, more than 65 million people are currently living with an incurable sexually transmitted disease (STD). An additional 15 million people become infected with one or more STDs each year, roughly half of whom contract lifelong infection. Cates, 26 Sex. Trans. Dis. S2-S7 (1999); CDC, Tracking the hidden epidemics: Trends in STDs in the U.S. (2000). Yet, STDs are generally an under-recognized health problem. Gonorrhoeae is one of the most frequently reported STDs in the United States. The reported rate of gonorrhea in the United States remains the highest of any industrialized country and is roughly 50 times that of Sweden and eight times that of Canada. CDC, 2000. Additionally, studies have shown that gonorrhea can facilitate HIV transmission and may be contributing significantly to the spread of HIV in parts of the U.S. and the world. There is a critical need to reach populations that remain at high risk for gonorrhea with intensified prevention. Therefore, the need for a vaccination against gonococcal diseases has arisen with the high incidences and the closely associations of HIV infection.

N. gonorrhoeae is a Gram-negative diplococcus that lacks a polysaccharide capsule. In the past, the search for gonococcal vaccine candidates has focused on surface-exposed outer-membrane components, such as major outer membrane proteins (MOMP), pili and lipo-oligosaccharide (LOS). The biological properties and antigenic variations of gonococcal major outer-membrane proteins (PorB, Rmp and Opa), pili and LOS have been well documented. PorB is a major protein constituent of the outer membrane and has moderate antigenic variability. Two subtypes of PorB are expressed by two porB genes that are mutually exclusive at the same genetic location of gonococcal genome. Gonococcal Rmp is a reduction-modifiable protein, similar to the OmpA-like proteins, which are found in all Gram-negative bacteria. Rmp protein has less antigenic variability, but the antibody against Rmp might block the complement-dependent bactericidal activity. Rice et al., 164 J Exp Med. 1735-48 (1986). Opa is a family of variant outer-membrane proteins associated with opaque colony phenotype. Pili are hair-like appendages that facilitate distant attachment of gonococci to eukaryotic cells. Expressions of Opa and Pili are subject to significant phase and antigenic variations. The problem of either an Opa- or Pili-based vaccine is the huge number of antigenic variants resulting from genetic exchanges or gene-conversion events. LOS is the major non-protein antigen of gonococci on the outer-membrane surface. Expression of LOS is also subject to phase and antigenic variations. In addition, toxicity of LOS endotoxin may limit the application of purified LOS molecules to vaccines.

By way of additional background, N. meningitidis and N. gonorrhoeae are the two pathogenic species of the genus Neisseria. Overall DNA homology between the two species is high: up to 77%. Guibourdenche et al., 137B Ann. Inst. Pasteur Microbiol. 177-85 (1986). Interspecies sequence similarity is increased to 98% in the “house-keeping genes.” Zhou & Spratt, 6 Mol. Microbiol. 2135-46 (1992). These two closely related species are associated with two distinct clinical spectra of diseases, however: N. gonorrhoeae infections are typically confined to the mucosa of the urogenital tract, with invasion of bloodstream occurring only rarely; whereas N. meningitidis colonizes the mucosal surface of nasopharynx and can invade the bloodstream, causing septicemia. N. meningitidis can also invade the central nervous system, causing life-threatening meningitis. N. meningitidis is divided into thirteen serogroups based on the biochemical and antigenic diversity of capsular polysaccharide antigens. Serogroup A, B, and C strains are responsible for more than 90% cases of meningococcal diseases in the world. The whole genome sequences of these three serogroups have been determined in the meningococcal reference strains (strain Z2491, serogroup A, MC58, serogroup B, and FAM18, serogroup C). Parkhill et al., 404 Nature, 502-06 (2000); Sanger Institute, on-line resources; Tettelin et al., 278 Science, 1809-15 (2000). The genome sequence of gonococcal reference strain FA1090 has also been completed and is available on-line from the University of Oklahoma.

In the 1960s, vaccines consisting of purified capsular polysaccharide antigens were developed against four (A, C, Y, and W135) of the five pathogenic serogroups of N. meningitides. Gotschlich et al., 129 J. Exp. Med. 1349 (1969); Gotschlich et al., 129 J. Exp. Med. 1367-84, (1969). Additionally, a vaccine consisting of outer membrane proteins against serogroup B N. meningitidis has been investigated, and apparently elicits serum bactericidal antibody responses and protects against developing meningococcal disease in clinical trials. Bjune et al., 338 Lancet, 1093 (1991); de Moraes et al., 340 Lancet, 1074-78 (1992); Tappero et al., 281 JAMA, 1520-27 (1999); Pizza et al., 287 Science, 1816-20 (2000).

The outer membrane adhesin/invasin protein OpcA shows strong evidence that it contributes significantly to the protective immunity of N. meningitidis serogroup B vaccine. Jolley et al., 69 Infect. Immun. 3809-16 (2001); Rosenqvist et al., 63 Infect. Immun. 4642-52 (1995). It was previously thought that the opcA gene was specific to N. meningitidis and lacking in N. gonorrhoeae. This supposition was disproved, however, by the discovery of opcA in the genomes of N. gonorrhoeae. Zhu et al., 33 Mol. Microbiol. 635-50 (1999). Comparison of the deduced peptide sequences from two representative N. gonorrhoeae strains, two N. polysaccharea strains, two representative N. meningitidis strains and thirteen N. meningitidis sequence variants revealed interspecies diversity of the OpcA protein family with conserved transmembrane regions and species-specific polymorphism at the surface-exposed loops and periplasmic turns. Zhu et al., 307 Gene, 31-40 (2003). Although the N. meningitidis opcA gene is more conserved, before the advent of the present invention there were only two opcA genes available in databanks. The ambitious sequencing study reported herein revealed that opcA is also conserved in the gonococci, suggesting less antigenic variability within the N. gonorrhoeae species than was thought previously. Therefore, as described herein, the OpcA protein is a potential candidate for the vaccine development targeting gonococcal disease.

As noted, N. meningitidis, the opcA gene is more conserved than other genes coding for outer membrane proteins. Seiler et al., 19 Mol. Microbiol. 841-56 (1996). More specifically, sequences of 1148 bp fragments that contain the opcA gene plus the flanking intergenic region from forty-three strains of serogroup A, B, C 29E, W-135, X and Y revealed eighteen distinct alleles, ten of which contained a 230 bp insertion in the 5′-flanking region. Within the 1148 bp nucleotide fragment, forty-eight (4.2%) sites were polymorphic: Twenty-three sites within the 819 bp coding region (2.8%), Twenty-four sites in the 293 bp 5′-flanking region (8.2%) and one polymorphic site in the 36 bp 3′-flanking region. Of the twenty-three base changes within the coding region, thirteen led to changes in the deduced protein sequence and one base change introduced an amber stop codon, leading to thirteen OpcA variants at the protein level. A total of 108 meningococcal strains were examined for the presence and absence of opcA by DNA hybridization analysis. The results showed that most meningococcal strains (serogroup A and ET-5 complex) contain the opcA gene, but strains of certain clonal group (ET-37 complex and cluster A4) and few random endemic isolates lack the opcA gene. Id. Hence, an aspect of the present invention provides for comparable insight into the conservation of the gonococcal opcA genes.

The nature of the N. meningitidis OpcA protein is well characterized, and of interest as a comparative model for the present invention. As noted previously, OpcA is an integral outer membrane protein from N. meningitidis, the causative agent of meningococcal meningitis and septicemia. N. meningitides bacteria isolated from the nasopharynx of patients and healthy carries expressed large amounts of OpcA more often than the isolates from cerebrospinal fluid or blood. Achtman et al., 164 J. Infect. Dis. 375-82 (1991). This suggests that OpcA might be important for adhesion to epithelial cells, and down-regulated variants might be selected during invasion. Indeed, strains lacking pili and capsular polysaccharide could adhere to and invade human endothelial and epithelial cells when expressing large amount of the OpcA protein. Virji et al., 6 Mol. Microbiol. 2785-95 (1992). The protein is believed to mediate the adhesion of N. meningitidis to epithelial and endothelial cells by binding to vitronectin and proteoglycan cell-surface receptors.

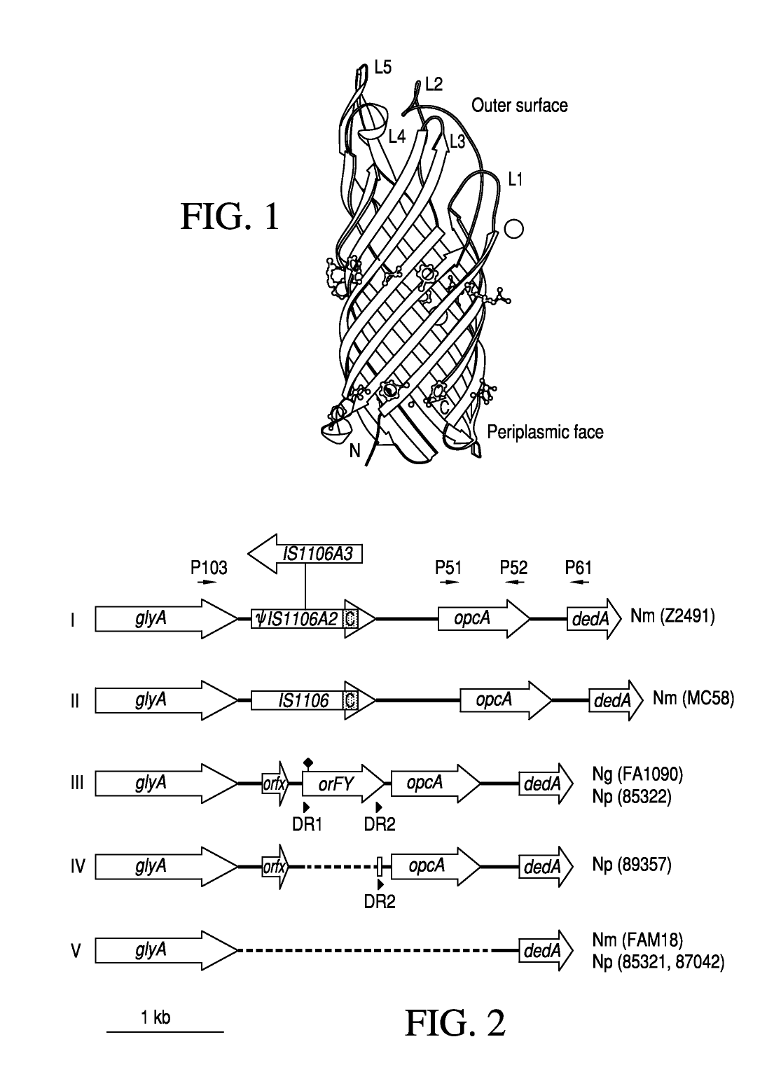

A two-dimensional structural model of meningococcal OpcA protein was devised by Merker et al. in 1997. See Merker et al., 16 Annu. Rev. Cell Dev. Biol. 423-57 (2000). The complete three-dimensional structure of meningococcal OpcA protein was constructed previously (FIG. 1). Prince et al., 2002; Riou & Guibourdenche, 37 Int'l J. Syst. Bacteriol. 163-65 (1987). OpcA adopts a ten-stranded beta-barrel structure with extensive loop regions that protrude above the predicted surface of the membrane (FIG. 1). The second external loop (Loop 2) adopts an unusual conformation, traversing the axis of the beta-barrel and apparently blocking formation of a pore through the membrane. Loop 2 is the largest loop and its tip was recognized by three monoclonal antibodies named 279/5c (Rosenqvist et al., 63 Infect. Immun. 4642-52 (1995)), A222 (Parkhill et al., 2000), and B306 (Achtman et al., 168 J. Exp. Med. 507-25 (1988)). Loops 4 and 5 contribute to a cell-surface-exposed epitope recognized by monoclonal 154D11 (Merker et al., 2000). Loops 2, 3, 4, and 5 associate to form one side of a crevice in the external surface of the structure, the other side being formed by Loop 1. The crevice is lined by positively charged residues and form an ideal binding site for proteoglycan polysaccharide. The structure, therefore, suggests a model for how adhesion of this important human pathogen to proteoglycan is mediated at the molecular level. Prince et al., 2002; Riou & Guibourdenche, 1987. Hence, an aspect of the present invention provides for structural characterization of the gonococcal OpcA, further bolstering its promise as a gonococcal vaccine. Therefore, an embodiment of the present invention provides for determination of the sequence variations of the opcA gene in diverse N. gonorrhoeae strains.

In N. gonorrhoeae, the nucleotide sequences of the opcA gene were determined from two reference strains FA1090 and MS11. Zhu et al., 34 FEMS Immunol. Med. Microbiol. 193-200 (2002). Three polymorphic sites, two synonymous mutations and one non-synonymous mutation, differed between the opcA of the two gonococcal strains, and a one codon deletion was found in MS11 opcA. The homology of the opcA genes was 59% between N. gonorrhoeae and N. meningitidis. Twenty-six strains of N. gonorrhoeae were examined by PCR using gonococcal opcA primer pairs. All yielded a product of the same size as FA1090 and opcA is therefore present in these gonococcal strains. Zhu et al., 33 J. Clin. Microbiol. 458-62 (1995); Zhu et al., 2002. Fifty-one N. gonorrhoeae strains, which were used for the lgt locus analysis (Zhu et al., 203 FEMS Microbiol. Lett. 173-77 (2001)), were examined for opcA by PCR and DNA hybridization. All strains showed presence of opcA in their genomes. The PCR products from the opcA gene were digested using four frequent-cutting restriction endonucleases (PCR-RFLP). The same PCR-RFLP patterns as that of reference strain FA1090 were observed in all gonococcal strains tested, showing conserved sequence of the opcA gene in N. gonorrhoeae. The fifty-one gonococcal strains were reference strains and a variety of clinical isolates including urethritis, disseminated gonococcal infection. Thus, the conclusion is that the opcA gene is present in most N. gonorrhoeae strains.

The opcA gene is rare in commensal Neisseria species. Recent results demonstrated that only 2/13 of N. polysaccharea strains (strains 85322 and 89357) contain the third orthologous opcA, N. polysaccharea opcA, distinct from the known opcA from N. gonorrhoeae and N. meningitides. Zhu et al., 2002. The N. polysaccharea-opcA identified in this study is closely related to N. gonorrhoeae-opcA (93%), but significantly different within the regions coding for most surface-exposed loops. The opcA gene was not detected in the other twenty-seven commensal Neisseria strains tested, which suggests that most commensal Neisseria species may lack an opcA gene. Zhu et al., 1999

The opcA gene may be located on a DNA island that may have been imported into Neisseria from another bacterial source. Comparative analysis showed at least five types of genetic organization at the opcA locus in Neisseria (Types I to V) (FIG. 2). Types I, II, III and V were previously reported in N. meningitidis and N. gonorrhoeae (Parmar et al., 15 Vaccine, 164151 (1997); Toleman et al., 3 Cell. Microbiol. 33-44 (2001); Zhou et al., 1995; Zhou et al., 2002), and Type IV is a novel organization observed for N. polysaccheae (Zhou et al., 1999). The opcA locus is flanked by two housekeeping genes, glyA and dedA and the composition between these two genes are hypervariable. N. meningitidis species possess three types of organization at the opcA locus, represented by N. meningitidis serogroup A, ST-4 complex/subgroup IV-1 strain Z2491 (Type I) (Parmar et al., 1997; Zhou et al., 2002), serogroup B, ST-32 complex/ET-5 complex strain MC58 (Type II) (Toleman et al., 2001), and serogroup C, ST-11 complex/ET-37 complex strain FAM18 (Type V) (Zhou et al., 1995; Zhou et al., 2003). Both Type I and Type II contain an N. meningitidis-opcA but differ by insertion or deletion of IS1106 element at the upstream region of opcA. N. meningitidis-opcA has a promoter containing a poly-C tract, and the variable expression of N. meningitidis-opcA is due to size variation of the poly-C tract. Seiler et al., 1996.

In contrast, the promoter with the poly-C tract and IS1106 element was not observed upstream of opcA in N. gonorrhoeae (Zhou et al., 1999; Zhou et al., 2003) (FIG. 2). Two genes with unknown function, orfX and pseudo-orfY, were located at the upstream region of opcA in N. gonorrhoeae (Type III). In N. polysaccharea species, two strains 85322 and 89357 belong to the Type III and IV genetic organization at the opcA locus, respectively. The upstream region of opcA in N. polysaccharea strain 85322 is similar to the Type III of N. gonorrhoeae but has no frameshift mutation in orfY. Most of orfY has been deleted in N. polysaccharea strain 89357 (Type IV) (FIG. 2). Alternatively, orfY might also be an insertion into the upstream region of opcA in N. gonorrhoeae and N. polysaccharea rather than a deletion, as a putative target site of 9 bp was observed. In addition, the two N. polysaccharea strains, 85321 and 87042, possess a deletion including opcA and the upstream region between glyA and dedA similar to that in N. meningitidis sergroup C, ST-11 complex/ET-37 complex strain FAM18 (Type V) (FIG. 2). The N. polysaccharea Type V strains might be an ancestral and Nm ST-11 complex/ET-37 complex and ST-8 complex/A4 cluster strains lacking opcA might acquire the deletion from N. polysaccharea. Taha et al., 28 Mol. Microbiol. 1153-63 (1998).

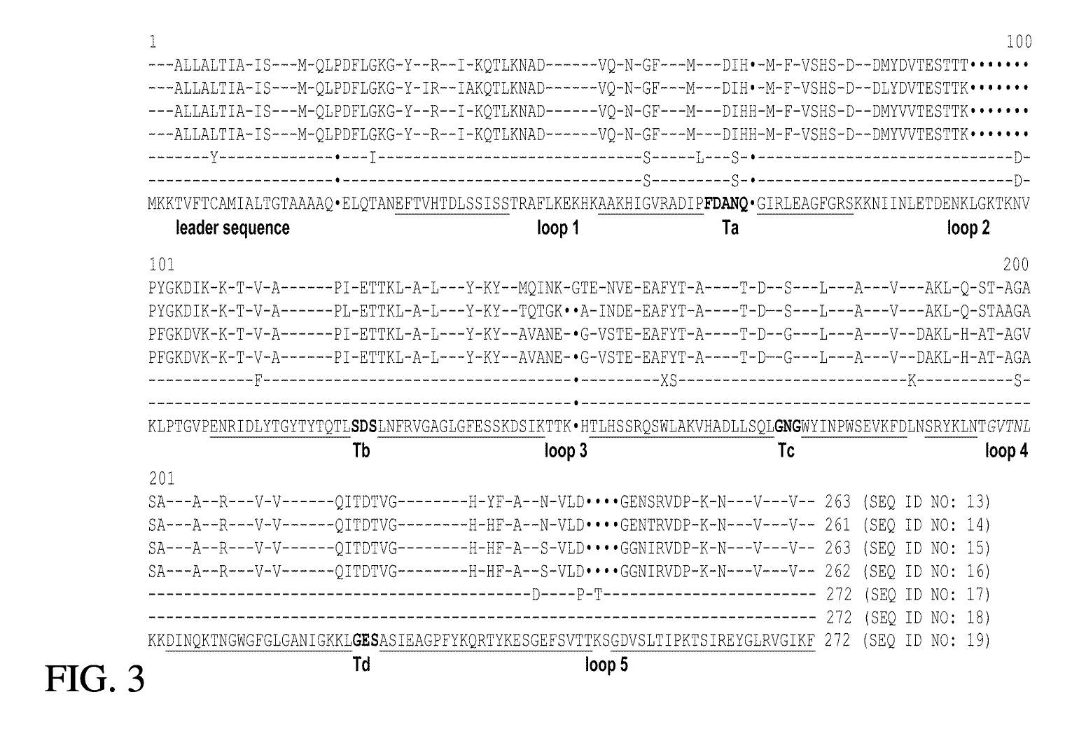

Comparison of the deduced peptide sequences from two representative N. gonorrhoeae strains, two N. polysaccharea strains, two representative N. meningitidis strains and thirteen meningococcal sequence variants demonstrates interspecies diversity of the OpcA protein family with conserved transmembrane regions and species-specific polymorphism at the surface-exposed loops and periplasmic turns. Zhu et al., 1999. Seventeen polymorphic sites are species-distinguishable among N. gonorrhoeae, N. meningitidis and N. polysaccharea in OpcA protein family (FIG. 3). Sixteen species-specific polymorphisms were located at the surface-exposed loops (loops 2, 3 and 4) and periplasmic turns (Tb and Tc) of the N. meningitides OpcA 3-D structure. Prince et al., 2002; Riou & Guibourdenche, 37 Int'l J. Syst. Bacteriol. 163-65 (1987). With reference to the results observed, it is interesting to consider the distribution of opacity (Opa) proteins within the Neisseria. Specifically, Opa adhesins are expressed in commensal Neisseria, as well as N. gonorrhoeae and N. meningitidis. Tramont et al., 68 J. Clin. Invest.:881-88 (1981). The binding specificity for human CEACAM1 is apparently conserved, although portions of the external loop regions in the commensal Opas tended to be shorter. It would therefore be of interest to know whether the biological functions of N. meningitidis-OpcA, adhesion to proteoglycan and vitronectin (de Vries et al., 27 Mol. Microbiol. 1203-12 (1998); Virji et al., 10 Mol. Microbiol. 499-510 (1993)), is preserved in N. gonorrhoeae-OpcA or N. polysaccharea-OpcA.

Comparison of the N. gonorrhoeae-OpcA, and N. polysaccharea-OpcA amino acid sequences against the 3-D crystal structure of N. meningitidis-OpcA suggests that these proteins may be able to form stable, folded, outer membrane proteins (FIG. 3). Zhou et al., 1999. Additionally, RNA analysis showed that both opcA genes from N. gonorrhoeae and N. polysaccharea were transcribable (id.; Zhou et al., 2003), although the level of expression of N. gonorrhoeae-OpcA was not strong in vitro. Moreover, bacterial protein expression can be triggered by contact with eukaryotic cells (Pizza et al., 2006; Tappero et al., 1999), or by conditions that are unique within an animal host (Camilli & Mekalanos, 18 Mol. Microbiol. 671-83 (1995); Hensel et al., 269 Science, 400-03 (1995); van Putten & Paul, 14 EMBO, 2144-54 (1995)), and it is conceivable that N. gonorrhoeae-OpcA is expressed efficiently during urogenital infections.

In characterizing the vaccine potential of the OpcA protein, the opcA genes were sequenced in the selected bacterial strains representative of maximum diversity of the natural population of N. gonorrhoeae; the recombinant OpcA protein was obtained by cloning and expression of gonococcal opcA gene in E. coli; systemic and mucosal immune responses were induced in mice using the recombinant OpcA protein; the potential protective effects of mouse anti-OpcA antibodies to both homologous and heterologous gonococcal strains were evaluated; the biological function of the opcA gene in N. gonorrhoeae was demonstrated.

The presence or absence of the opcA gene in a total of 210 N. gonorrhoeae strains was analyzed by PCR and DNA sequencing in the present invention. Two internal primers, O1 (5′-CATTGCTTGCACTGACTATT-3′) (SEQ ID NO:1) and O2 (5′-AGGATCGACTCGGATGTTCC-3′) (SEQ ID NO:2), were used to screen the presence and absence of the opcA gene in the N. gonorrhoeae strains. These strains included 100 diverse gonococcal strains and two reference strains (FA1090 and MS11). The opcA genes in reference strains FA1090 and MS11 were previously described (Zhu et al., 1999). The DNA samples of the two reference strains were used as positive controls in all experiments. Initially, the opcA gene in these strains was examined using PCR with opcA-specific internal primers. The PCR results showed that the opcA gene was detected in all 210 strains examined (100%), indicating that the opcA gene is widely distributed in species N. gonorrhoeae.

Sequence diversity of the gonococcal opcA gene was also explored. Two external primers, Y1 (5′-GGAGTGCCGTTCAGAATCGT-3′) (SEQ ID NO:3) and OF2 (5′-ACCATCAAATGAATATCCAT-3′) (SEQ ID NO:4), were used to amplify a fragment of 1389 bp from the gonococcal strains. The PCR product contains a 792 bp of the entire opcA open reading frame plus a 235 bp of upstream region and a 362 bp of downstream region from N. gonorrhoeae. By using the opcA-flanking primers, a DNA fragment containing entire opcA gene was amplified by PCR from all 210 gonococcal strains. The DNA sequences of the opcA gene were determined from the 210 strains and the results were compared to those from the two reference strains, FA1090 and MS11. A total of fourteen polymorphic sites were identified within 792 bp of opcA coding region (1.77%). Six of them were previously described in the opcA genes from FA1090 and MS11. Two polymorphic sites were synonymous mutations, whereas the rest polymorphic sites were nonsynonymous mutations, resulting in amino acid substitutions in the OpcA protein. Two amino acid substitutions were at the surface Loop 2 of OpcA protein and one substitution was at the surface Loop 4. Other amino acid substitutions were at transmembrane regions or inner turns. These results showed that gonococcal opcA gene is a highly conserved gene, compared to other genes encoding outer-membrane proteins in the N. gonorrhoeae genome.

Cloning and expression of the gonococcal opcA gene used two primers, O13 and O10, designed to amplify the region coding for gonoccoccal OpcA protein of Ala¹⁹ to Phe²⁶³ (the mature protein omitting the signal sequence), and introduce BamH I and Hind III restriction sites for cloning into the pRSET-A expression vector (Invitrogen, Carlsbad, Calif.). The forward primer O13 was 5′-GCCGGATCC³⁰⁰⁰GCCCAGTTGCCCGACTTT³⁰¹⁷-3′ (SEQ ID NO:5), and the reverse primer O10 was 5′-CTGAAAGCTT³⁷³⁷TTAGAATTTCACGCCGAC³⁷²⁰-3′ (SEQ ID NO:6); the numbers refer to positions within the sequence of strain FA1090 (GenBank accession number AJ242839), underlining indicates the additional bases introduced for cloning, and the bold indicates the restriction sites.

The pRSET-A plasmid and the amplified 757-bp PCR product were digested with BamH I and Hind III restriction endonucleases (New England Biolabs, Ipswich, Mass.). The digested products were purified by QIAquick spin-column. The opcA-containing fragment was ligated into pRSET-A using T4 DNA ligase (New England Biolabs), and the ligation mixture was used to transform competent E. coli TOP10F′ and selected on LB plates containing 100 μg/ml ampicillin. One such plasmid construct pOPC-R11 was purified by using QIAprep Miniprep Kit (Qiagen). The insertion of the opcA gene in the plasmid pOPC-R11 was confirmed by PCR and DNA sequencing. PCR amplifications with different primer combinations produced amplicons with expected sizes, suggesting the opcA gene was cloned into the expression vector with correct orientation. Further results from DNA sequencing revealed that no mutation occurred during the cloning step, and therefore the recombinant protein encoded by the pOPC-R11 has the exact protein sequence as its original OpcA in gonococcal strain FA1090.

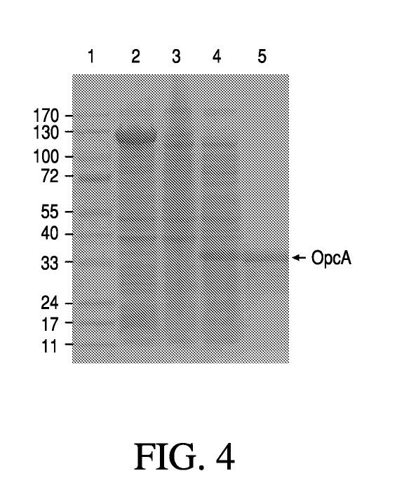

An embodiment of the present invention provides for the expression and purification of recombinant gonococcal OpcA protein. To express recombinant OpcA protein, the plasmid pOPC-R11 was transformed into E. coli BL21(DE3)pLysS cells (Invitrogen) and selected on LB agar containing 35 μg/ml chloramphenicol and 50 μg/ml ampicillin. A pilot expression of recombinant OpcA protein was performed and an optimal condition was used for maximizing the OpcA protein yields. Briefly, a single colony of recombinant E. coli was inoculated into 2 ml of SOB containing 35 μg/ml chloramphenicol and 50 μg/ml ampicillin, and grown at 37° C. with shaking of 225 rpm overnight. One (1) ml of overnight culture was inoculated into 50 ml of the SOB and grown with shaking to OD₆₀₀=˜0.5, 0.5 ml of 100 mM IPTG was added and grown at 37° C. with shaking for 4 hr. The cells were collected by centrifugation at 3,000×g for 10 min at 4° C. His-tagged recombinant OpcA protein was purified using Probond Nickel-Chelating Resin (Invitrogen) under hybrid conditions. The hybrid conditions combined the protocols to prepare bacterial lysate and affinity column under denaturing conditions and then use native buffers during the wash and elution steps to refold the protein. The eluted protein from the resin column was dialyzed against 10 mM Tris, pH 8.0, 0.1% Triton X-100 overnight at 4° C. to remove the urea. The protein solution was then concentrated at 5,000×g by ultrafiltration through an YM-10 Centricon Centrifugal Filter with a 10,000 MW cut-off membrane (Millipore). The concentration of recombinant OpcA protein was determined by using the BCA protein assay (Pierce). SDS-PAGE analysis showed only a single protein band about 31 KDa (FIG. 4), which was consistent with predicted size of fusion OpcA protein with His tag (31.2 KDa) based on the amino acid sequence.

Five different preparation of recombinant OpcA antigens with or without adjuvant were used for mouse immunizations. The recombinant OpcA protein (rOpcA) was first dissolved into PBS to a concentration of 2 mg/ml. The OpcA antigen solutions were prepared for mouse immunization as follows:
Group I (rOpcA): The protein solution was diluted with PBS to give a final concentration of 1 mg/ml.
Group II (rOpcA-CTB): The OpcA solution (2 mg/ml) was mixed with an equal volume of 1 mg/ml cholera toxin subunit B (Sigma, St. Louis, Mo.) to a final concentration of 1 mg/ml OpcA and 1 mg/ml CTB.
Group III (rOpcA-Liposome): The recombinant OpcA protein was incorporated into liposome by dialysis-sonication as described. Muttilainen et al., 18 Microb. Pathog. 18:423-436 (1995); Samuel et al., 75. Int'l J. Cancer. 295-302 (1998). Briefly, L-α-phosphatidylcholine and cholesterol (Sigma) combined at a 7:2 molar ratio (total 25 mg) and dissolved in chloroform at a concentration of 10 mg/ml (w/v) in a 250 ml glass round-bottom flask, and solvent was removed under vacuum in a rotatory evaporator (VV Micro, Heidolph Instrument) to produce an even lipid film. The recombinant OpcA protein (1 mg) was dissolved in a solution containing a final concentration of 0.1% SDS (w/v), 10 mM HEPES (pH 7.2) and 10% octylglucoside (w/v). This detergent-protein solution was used to solubilize the shell dried lipid. The mixture was then extensively dialysed in a cellulose tube (MWCO 12,000-14,000; Fisher Scientific) against PBS (pH 7.2) at 4° C. The resulting milky solution was subject to repeated 10 sec bursts of ultrasonication (Sonifier 250, Branson) on ice to produce small unilamellar membrane vesicles. Liposomes were then collected by centrifugation at 100,000×g for 1 hr and finally reconstituted to the desired volume.
Group IV (rOpcA-micelle): 4 mg of recombinant OpcA protein was dissolved in 1 ml of 50 mM Trisbuffer (pH 8.0) containing 100 mM NaCl and 0.2% SDS (w/v). This was used as a stock solution with concentration of 4 mg/ml OpcA. Three (3) ml of 0.2% SDS containing 32 mg of Zwittergent 3-14 (Calbiochem) was added to the stock solution to a final concentration of 1 mg/ml rOpcA, 0.2% SDS and 8 mg/ml Zwittergent. The mixture was incubated at room temperature overnight.
Group V (rOpcA-R700): The OpcA protein solution was diluted with PBS to a concentration of 0.2 mg/ml. A syringe with 20 gauge needle was used to inject this 2 ml of rOpcA solution into a vial of R-700 (Ribi Immunochem) through the rubber stopper, to a final concentration of 0.1 mg/ml rOpcA. The vial was vortexed vigorously for 3 min. The emulsified OpcA antigen with R-700 Ribi adjuvant given a final concentration of 10 μg OpcA protein per 100 μl emulsion for mouse immunization according to the manufacturer's instructions.

OpcA Immunizations and sample collections. BALB/c female mice at six to seven weeks of age were used for intranasal immunization. Five groups of mice, ten mice each, were immunized either intranasally or subcutaneously with 20 μl of recombinant OpcA protein alone, associated with different adjuvants or liposome as shown in Table 1. Mice were immunized on days 0, 14, 28 and 46 for the intranasal route, and days 0, 21, and 42 for the subcutaneous route. Blood samples were taken on day-0 and day-35, and the mice were terminally bled on day-60, and sera were stored at −20° C. Vaginal washes were collected on days 0, 35 and 60 bp repeated flushing and aspiration of 30 μl of PBS containing 0.1% BSA (Sigma) and 1 mM phenylmethylsulfonyl fluoride (Sigma) as protease inhibitor. The vaginal washing was repeated twice and the fluid specimens were pooled and stored at −20° C.

TABLE 1
Mouse immunization groups

| | Immunization | | | Volume | Amount | | Sera and vaginal |
|---|---|---|---|---|---|---|---|
| Group | Route | Immunogen | Conc. | (μl) | (μg) | Day admin. | fluid collection |
| I | Intranasal | rOpcA | 1 mg/ml | 20 | 20 | 0, 14, 28, 46 | 0, 35, 60 |
| II | Intranasal | rOpcA- | 1 mg/ml + | 20 | 20 + 10 | 0, 14, 28, 46 | 0, 35, 60 |
| | | CTB | 0.5 mg/ml | | | | |
| III | Intranasal | rOpcA- | 1 mg/ml | 20 | 20 | 0, 14, 28, 46 | 0, 35, 60 |
| | | liposome | | | | | |
| IV | Intranasal | rOpcA- | 1 mg/ml | 20 | 20 | 0, 14, 28, 46 | 0, 35, 60 |
| | | micelle | | | | | |
| V | Subcutaneously | rOpcA- | 0.1 mg/ml | 200 | 20 | 0, 21, 42 | 0, 28, 60 |
| | | R700 | | | | | |

The antibody responses in mouse sera were measured by using ELISA. Sera samples were collected from pre-immune (day-0) and post-immune (day-35 and day-60). All pre-immune samples were assayed and found to be negative for antibodies specific to OpcA protein tested. OpcA-specific antibody was detected in the sera collected at day-35 after the first OpcA immunization and day-60 after the second and third OpcA immunizations as shown in Table 2. Serum immune response show consistent profiles of three immunoglobulin classes IgG, IgA and IgM in all five immunization groups. In general, anti-OpcA antibody response was observed in groups II (OpcA-CTB), IV (OpcA-micelle) and V (OpcA-R700). The strongest IgG antibody response was induced by subcutaneous immunization of the OpcA protein with R700 adjuvant. The OpcA protein induced only weak antibody response when administered intranasally to mice in the absence of adjuvant or in presence of liposome, however. The data indicated that the adjuvant has different enhancing effect on the OpcA immunization. Groups II, IV and V had the IgG antibody levels which were 2 log₁₀ to 3 log₁₀ units higher than those in mice immunized OpcA only or OpcA adjuvanted with liposome. There was no significant difference IgA and IgM level between different groups. The IgM antibody level was generally decreased between day-35 and day-60 in all immunization groups.

TABLE 2
Anti-OpcA antibody response in mouse serum samples

| | Immunization | | Day-35 Sera (GMT ± SE) | | | Day-60 Sera (GMT ± SE) | | |
|---|---|---|---|---|---|---|---|---|
| Group | Route | Immunogen | IgG | IgA | IgM | IgG | IgA | IgM |
| I | Intranasal | rOpcA | 673 ± 1074 | 109 ± 35 | 617 ± 207 | 2,934 ± 8308 | 130 ± 52 | 141 ± 53 |
| II | Intranasal | rOpcA- | 16127 ± 3355 | 379 ± 481 | 635 ± 400 | 55299 ± 58346 | 1481 ± 1116 | 343 ± 88 |
| | | CTB | | | | | | |
| III | Intranasal | rOpcA- | 187 ± 459 | 107 ± 32 | 246 ± 97 | 800 ± 413 | 115 ± 42 | 200 ± 103 |
| | | liposome | | | | | | |
| IV | Intranasal | rOpcA- | 19740 ± 3657 | 238 ± 212 | 566 ± 214 | 51200 ± 28043 | 264 ± 241 | 303 ± 110 |
| | | micelle | | | | | | |
| V | Subcu. | rOpcA- | 135118 ± 68267 | 115 ± 42 | 3200 ± 1652 | 289631 ± 205084 | 119 ± 50 | 1213 ± 775 |
| | | R700 | | | | | | |

Mucosal immune responses against gonococcal OpcA protein were analyzed. The antibody responses in mouse vaginal wash were measured by using ELISA. Mouse vaginal wash samples were collected from preimmune (day-0) and post-immune (day-35 and day-60). All pre-immune samples were assayed and found to be negative for antibodies specific to OpcA protein tested. In contrast to serum immune response, mucosal immune response show different profiles of OpcA-specific immunoglobulin class. Though group V has the highest anti-OpcA IgG antibody level in vaginal wash, its IgA antibody response was the lowest in all five groups. Strong IgA antibody response to OpcA was observed in intranasal immunization groups IV and II. The data suggests that intranasal immunization could induce mucosal IgA immune response to gonococcal OpcA protein. IgM antibody response was weak or undetectable in vaginal wash samples, as shown in Table 3:

TABLE 3
Anti-OpcA antibody response in mouse vaginal wash samples

| | Immunization | | Day-35 Sera (GMT ± SE) | | | Day-60 Sera (GMT ± SE) | | |
|---|---|---|---|---|---|---|---|---|
| Group | Route | Immunogen | IgG | IgA | IgM | IgG | IgA | IgM |
| I | Intranasal | rOpcA | 46 ± 129 | 77 ± 121 | ≦25 ± 0 | 65 ± 58 | 65 ± 58 | ≦25 ± 0 |
| II | Intranasal | rOpcA- | 63 ± 70 | 100 ± 112 | 27 ± 8 | 432 ± 682 | 272 ± 224 | ≦25 ± 0 |
| | | CTB | | | | | | |
| III | Intranasal | rOpcA- | 71 ± 65 | 93 ± 246 | 27 ± 8 | 66 ± 30 | 81 ± 71 | ≦25 ± 0 |
| | | liposome | | | | | | |
| IV | Intranasal | rOpcA- | 109 ± 50 | 183 ± 118 | ≦25 ± 0 | 51200 ± 28043 | 919 ± 1200 | ≦25 ± 0 |
| | | micelle | | | | | | |
| V | Subcu. | rOpcA- | 1838 ± 2223 | 38 ± 24 | ≦25 ± 0 | 4850 ± 4681 | 33 ± 24 | ≦25 ± 0 |
| | | R700 | | | | | | |

Subclass distribution of anti-OpcA IgG antibody was also examined. The subclass of IgG that is induced after immunization is an indirect measure of the relative contribution of TH2-type versus TH1-type responses. The production of IgG1 antibodies is primarily induced by TH2-type, whereas production of IgG2a antibodies reflects the involvement of TH1-type. The effect of different immunization approaches on the TH1 and TH2 profile was assessed by determining the ratio of IgG1 to IgG2a anti-OpcA antibody. Mouse IgG2a is considered as the most efficient subclass in activating complement, while IgG1 is poor and may be inhibitory. Ey et al., 17 Mol. Immunol. 699-710 (1980); Koolwijk et al., 28 Mol. Immunol. 567-76 (1991); Seino et al., 94 Clin. Exp. Immunol. 291-96 (1993). Herein, the analysis of the IgG subclass of the anti-OpcA antibody in serum sample revealed that both IgG1 and IgG2a were induced following immunization of the OpcA protein either with or without adjuvant. The IgG1 antibody was the dominant subclass in all immunization groups. Mouse Group I, immunized with OpcA alone, had the highest IgG1 response (74.9%), therefore a higher IgG1/IgG2a ratio (12.2). Interestingly, mouse IgG2a response was increased by immunization of OpcA in combination with the adjuvants, which reduced the IgG1/IgG2a ratio to 1.4-1.8 in other four immunization groups. These data suggest that incorporation of adjuvant with the OpcA antigen had an effect on the subclass response of IgG antibody. In addition, bactericidal assays detected bactericidal activity from immunization Groups II and IV, which was consistent with an IgG1/IgG2a ratio reduction. Unexpectedly, the bactericidal assays did not detect a strong bactericidal activity from immunization Group V.

Bactericidal activity of anti-OpcA antibody was investigated because bactericidal antibodies are an important correlate of protection against gonococcal infection. Most current licensed vaccines have relied on the bactericidal assays to predict vaccine efficiency. In the present invention, antisera raised against recombinant OpcA protein were tested for their bactericidal activity against the homologous strain FA1090. Initially, all forty-two serum samples collected on day-60 were diluted with PBS to 1:16 and pre-screened for their bactericidal activity. Twenty-five of them (59.52%) showed bactericidal activity (>1:16) (Table 4), although seventeen of them did not show any bactericidal activity (<1:16). Bactericidal activity of the twenty-five positive samples was further titrated using serial dilutions of the pre-immune (day-0) control serum and post-immune (day-60) serum samples. The titration results of bactericidal activity are shown in Table 4:

TABLE 4
Bactericidal activity of antisera raised against recombinant OpcA protein

| | | | Mice | Mice | Positive^{b} | Bacterial GMT^{c} | |
|---|---|---|---|---|---|---|---|
| Group | Immunization | Immunogen | immunized | survived^{a} | >1:16 | A | B |
| I | Intranasal | rOpcA | 10 | 8 | 4 | 32.0 ± 0.00 | 24.7 ± 38.66 |
| II | Intranasal | rOpcA-CTB | 10 | 9 | 6 | 101.6 ± 99.15 | 54.9 ± 96.33 |
| III | Intranasal | rOpcA-liposome | 10 | 10 | 7 | 25.4 ± 7.81 | 21.1 ± 8.26 |
| IV | Intranasal | rOpcA-micelle | 10 | 5 | 5 | 152.2 ± 208.00 | 97.0 ± 204.40 |
| V | Subcu. | rOpcA-R700 | 10 | 10 | 3 | 128.0 ± 0.00 | 29.9 ± 54.10 |
| Total | | | 50 | 42 | 25 | | |

^{a}Number of mice surviving on day-60.
^{b}Number of day-60 samples with bactericidal titer >1.1:16.
^{c}Bactericidal geometric mean titers with standard error (GMT ± SE) are calculated from reciprocal dilution which produced >50% killing. “A” indicates an average titer of the samples from the positive mice (bactericidal titer >1:16). “B” indicates an average titer of serum samples from all survived mice each immunization group.

The antisera from immunization Group I (rOpcA only) and Group III (rOpcA-liposome) did not show significant bactericidal activity. The antisera from Group II (rOpcA-CTB), Group IV (rOpcA-micelle), and Group V (rOpcA-R700) showed bactericidal activity with the highest titer in the serum samples from Group IV (97.0±204.40). Because only half of the mice were alive in Group IV on day-60, the question of whether rOpcA-micelle was toxic to the mice may be studied further. Despite impressive serum antibody titers, antisera raised from subcutaneous approach were only weakly bactericidal (29.9±54.10) in Group V. In contrast, antisera from intranasal approach showed higher bactericidal activity in Group II and Group IV, indicating that intranasal delivery with mucosal adjuvant was effective generating bactericidal antibody in mice. Immunizations using either of the rOpcA-CTB or rOpcA-micelle formulations substantially increased the bactericidal activity of sera in mice.

A further embodiment of the present invention provides for the biological function of OpcA in N. gonorrhoeae. To determine the biological function of OpcA in the N. gonorrhoeae, an opcA knockout mutant was constructed by insertion of a kanamycin resistance cassette (Kan) into the N. gonorrhoeae genome. The insertion of Kan into the opcA gene of gonococcal genome was confirmed by multiple PCRs and Southern Blot Hybridization. This opcA-isogenic mutant was then used for cell adherence assays.

Construction of the opcA-isogenic mutant: In order to determine the biological function of gonococcal OpcA protein, an opcA-isogenic mutant has been constructed. The pOPC-R11 plasmid containing 738 bp of the opcA gene (positions 3000-3737 of AJ242839) was digested with restriction endonuclease Sph I, which cuts the opcA gene at position 209-214 after ATG start codon. The overhangs at the end of DNA fragment were filled in with T4 DNA polymerase (Promega), purified through QIAquick spin-column, and then added to Hinc II— digested kanamycin resistance cassette (Kan) from pUC4Kan (GE Healthcare, Piscataway, N.J.). The blunt ends were ligated using T4 DNA ligase (Promega, Madison, Wis.). After transformation into E. coli TOP10 cells and selection on LB agar plates containing 50 μg/ml kanamycin, colonies were examined by PCR for the constructs with the kanamycin cassette in the same transcriptional orientation as the opcA gene. One such plasmid construct, pOPC-RK13, was linearized by digestion with BamH I, and was used to transform strain FA1090 of N. gonorrhoeae by electroporation. Cells were plated upon GC agar plate containing 50 μg/ml kanamycin, and the transformants were confirmed as opcA mutants by PCR and Southern blot hybridization.

In the Southern blot hybridization assay, bacterial genomic DNA was isolated from the mutant and wild-type strain by using QIAamp DNA Mini Kit (Qiagen, Valencia, Calif.). The genomic DNAs were digested with BamHI, subjected to electrophoresis on a 0.7% agarose gel and transferred to a positively charged nylon membrane (Roche). The probe was generated from the opcA gene of strain FA1090 bp PCR using primers, O1 (5′-CACTGCTTGCACTGACTATT-3′) (SEQ ID NO:7) and O2 (5′-AGGATCGACTCGGATGTTCC-3′) (SEQ ID NO:8), and DIG PCR Probe Synthesis Kit (Roche). Hybridization and washes were performed using DIG Nucleic Acid Detection Kit (Roche) according to manufacture's instructions. In the PCR assays, the genomic DNA of the mutant FA1090ΔopcA and wild-type strain FA1090 were amplified by using two Kan-specific primers of P101 (5′-GACAATCTATCGATTGTATG-3′) (SEQ ID NO:9), P102 (5′-GTCCAACATCAATACAACCT-3′) (SEQ ID NO:10), and two opcA-specific primers (P299 and P300).

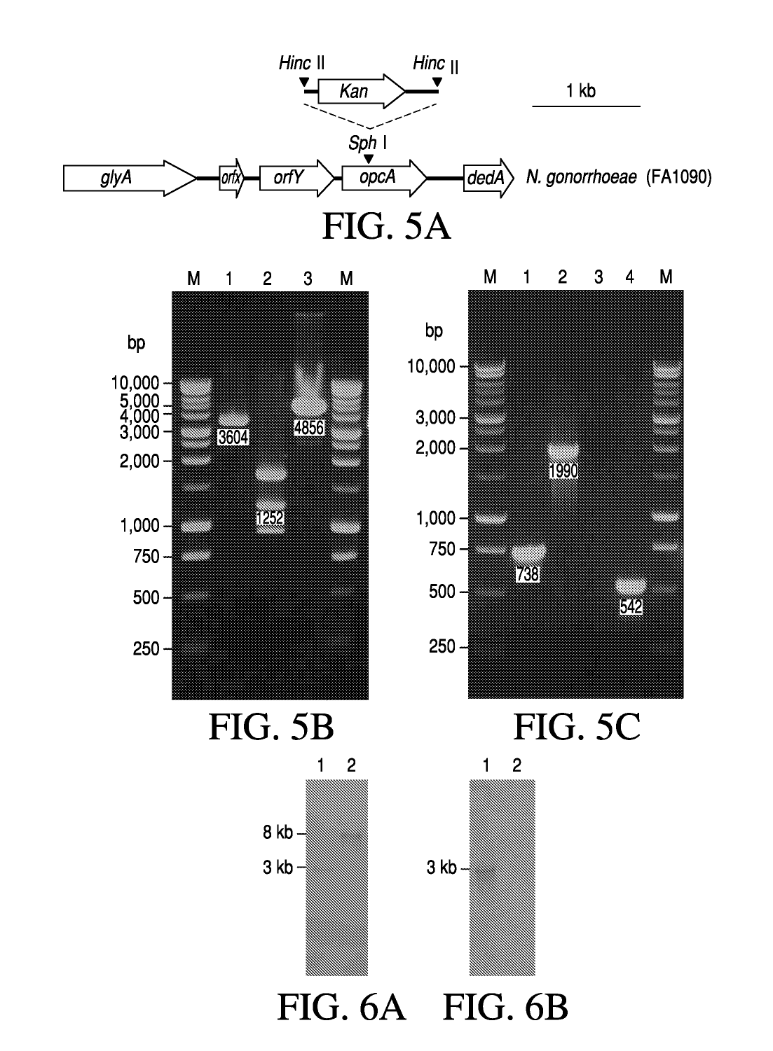

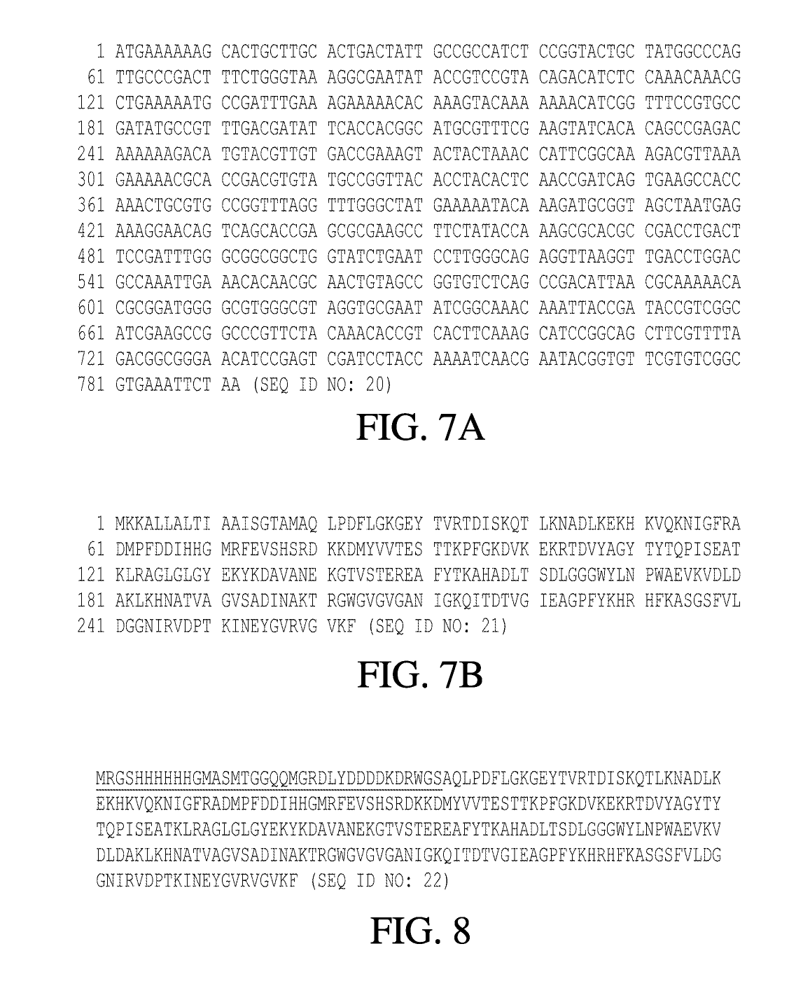

Confirmation of opcA-isogenic mutant was undertaken. To create a mutant that completely inactivates the opcA gene, a kanamycin resistance cassette (Kan) was introduced into the Sph I restriction site that is located at position 209-214 after the ATG start codon of the 807 bp coding region (FIG. 5A). First, a plasmid that contained a kanamycin resistance cassette (Kan) with the sequences flanking the opcA gene was constructed. Initial plasmid pOPC-R11 containing the opcA gene was digested with restriction endonuclease Sph I and ligated with a Kan DNA fragment derived from restriction endonuclease Hinc II digestion of plasmid pUC4kan. After transformation of ligation mixture into E. coli TOP10 cells and selection on LB agar containing ampicillin, the colonies were screened and verified by PCR and restriction analyses. One plasmid containing the final structure was designated pOPC-RK13. FIG. 5B shows the results of restriction analysis of the initial plasmids and final plasmid construct. The initial plasmid pOPC-R11 containing the opcA gene was digested with Sph I and yielded a 3064 bp of linearized DNA fragment (FIG. 5B, Lane 1). A plasmid pUC4Kan containing Kan was digested with Hinc II and yielded three DNA fragments (FIG. 5B, Lane 2). One of them (1252 bp fragment) contains the entire region of the Kan cassette. This Kan fragment was ligated into Sph I-digested pOPC-R11, which generated the final plasmid pOPC-RK13 of 4856 bp containing the expected construction (FIG. 6B, Lane 3). The construction of plasmid pOPC-RK13 was confirmed by DNA sequencing.

N. gonorrhoeae strain FA1090 was used for construction of the opcA isogenic mutants. Plasmid pOPC-RK13 containing the opcA gene with a Kan insertion was linearized by BamHI digestion and then transformed into strain FA1090, an isogenic opcA-deficient mutant was generated by allele exchange. One mutant, designated F1090ΔopcA, was analyzed by PCR to confirm the insertion of Kan into the opcA gene in the FA1090 genome. Genomic DNA from FA1090 wild-type strain and isogenic mutant were amplified by PCR, using primers corresponding to the sequences of the Kan cassette and the opcA gene regions, respectively (FIG. 5C). By using opcA-specific primers P299 and P300, FA1090 wild-type strain yielded a 738 bp amplicon of the opcA gene (FIG. 5C, Lane 1), whereas the F1090ΔopcA mutant yielded an amplicon about 1990 bp (FIG. 5, Lane 2). The difference between the sizes of the amplicons is consistent with the insertion of the Kan cassette, which is 1252 bp in size, into the opcA gene (FIG. 5C). This suggests that Kan has been inserted into the opcA gene in FA1090ΔopcA. By using Kan primers, a specific amplicon of 542 bp of the kan gene was obtained from the mutant F1090ΔopcA (FIG. 5C, Lane 4) but not from the wild-type FA1090 strain (FIG. 5C, Lane 3), indicating that the Kan gene has been integrated into the chromosome of mutant FA1090ΔopcA. All PCRs yielded amplicons of the predicted sizes, suggesting that Kan was inserted into the correct location of FA1090 genome.

Southern blot analysis was performed to further characterize the mutant. Genomic DNA from the isogenic mutant FA1090ΔopcA and wild-type FA1090 strain were digested with restriction endonuclease Hind III. Hind III-digested genomic DNAs from the mutant and the wild-type strain were probed with the opcA and kan probes, respectively (FIG. 6). The opcA probe bound to approximately 3 kb DNA fragments in the mutant (FIG. 6A, Lane 1) and 8 kb DNA fragment in the wild-type strain (FIG. 6A, Lane 2). This is consistent with the insertion of the Kan cassette, which contains a Hind III site at the position of 550-555 after the ATG start codon of Kan coding region resulting in a reduction size of bound-band in the mutant. The kan probe bound to 3 Kb DNA fragment of the mutant (FIG. 6B, Lane 1) but did not bind to wild-type DNA (FIG. 6B, Lane 2). The results of Southern blotting analysis further confirm that the opcA gene in the mutant FA1090ΔopcA has been successfully inactivated.

Immunofluorescence revealed additional characteristics. To prepare adherent bacteria for immunofluorescence staining, ME-180, a human cervical cancer cell line, was cultured in a Lab-Tek chamber slide (Nalge Nunc Int'l). The cell monolayers in the chamber were infected with FA1090 wild-type strain and opcA-isogenic mutant. After the incubation the bacterial suspension was removed. The cell monolayers were washed three times with PBS to remove the unbound bacteria. The cell monolayer was fixed in PBS containing 2.0% paraformaldehyde and 0.5% glutaraldehyde at room temperature for 20 min (Gill et al., 2003). The monolayers were incubated with 0.5% Triton X-100 in PBS at room temperature for 20 min to make the cells permeable for antibody detection. To stain gonocococii, the monolayers were first washed with PBS, and then incubated with rabbit polyclonal antibody against whole gonococci (Abcam, Cat# ab19962), at 37° C. for 60 min. After washing of cell layer with PBS, goat anti-rabbit antibody conjugated with fluorescein isothiocyanate (FITC) was added and incubated at 37° C. for 60 min. The cell monolayer was washed with PBS three times. The bacteria were counted using an Olympus fluorescence microscope. Bound bacteria were counted on 100 cells in each experiment. Adherence of the opcA-mutant was reduced compared to that of the wild-type strain. The intensity of ME-180 cells in the culture chamber also affected the ratio of bacteria/cells. When 2×10⁵ ME-180 cells were cultured in each chamber, adherence ratio of the opcA-mutant to ME-180 cells decreased about 34.7% compared to of the wild-type strain control. When 2×10⁴ ME-180 cells were cultured in each chamber, the ratio decreased about 21.8%. These results of immuno-staining assay were consistent with those of the real-time PCR assay. Both assays showed that inactivation of the opcA gene could reduce the bacterial adherence to human epithelial cells, suggesting that gonococcal OpcA protein is involved in pathogenesis.

The analysis of 210 strains representing the extensive diversity of the natural population of N. gonorrhoeae revealed that the sequence polymorphism of the opcA gene shows only fourteen sites (1.77%) are polymorphic in the 792 bp of opcA coding region. The data obtained support the original hypothesis that gonococcal opcA gene is more conserved than other OMP genes, indicating less antigenic variations of the OpcA protein in N. gonorrhoeae. Therefore, gonococcal OpcA protein has vaccine potential to induce a broad range of protection.

To summarize, the cloning and expression of the gonococcal opcA gene in E. coli was achieved, and recombinant OpcA protein purified. The recombinant OpcA protein was used for mouse immunizations. Both intranasal and subcutaneous immunizations of the OpcA protein were capable of generating local or systemic antibody responses, demonstrating that the OpcA protein is highly immunogenic to mouse. Bactericidal activity was induced when the OpcA protein was administered with CTB or micelle, suggesting that intranasal delivery with mucosal adjuvant was important in generating bactericidal antibody.

The instant invention also provides for functional analysis of gonococcal opcA gene. An opcA-isogenic mutant (FA1090ΔopcA) was constructed from the reference strain FA1090 bp using kanamycin resistance cassette of pUC4Kan to inactivate the coding region of the opcA gene. The insertion mutation of the opcA gene was confirmed by PCR, DNA sequencing and Southern blot. Adherence assays showed that inactivation of the opcA gene could reduce the adherence of N. gonorrhoeae to human ME-180 epithelial cells, suggesting that gonococcal OpcA protein acts as an adhesion between gonococci and eukaryotic cells. Additionally, the recombinant OpcA allows for the characterization of the OpcA epitopes that induce specific immune response and bactericidal activity and identification of OpcA domains important for bacterial adherence.

The immunogenic OpcA of the present invention may be harvested from N. gonorrhoeae. Immunogenic portions of OpcA may be produced by proteolytic digestion. The OpcA or portions thereof may also be produced using recombinant techniques, or by chemical synthesis, all of which are known in the art.

The OpcA peptides or proteins of this invention may be administered as multivalent subunit vaccines in combination with other antigens of N. gonorrhoeae or antigens of other organisms. Some of the other organisms include the pathogenic bacteria H. influenzae, N. meningitidis, S. pneumoniae, etc. For example, they may be administered in conjunction with Pili, Tbp, PorB, and/or LOS components of N. gonorrhoeae, or oligo- or polysaccharide capsular components of N. meningitidis or S. pneumoniae. Similarly, an immunogenic OpcA peptide may be conjugated to another hapten, thus acting as an effective protein carrier or adjuvant for that hapten. Hapten refers to a disease specific antigenic determinant identified by biochemical, genetic or computational means. The haptens may be associated with a disease condition caused by N. gonorrhoeae, or by an agent such as bacteria, viruses, intracellular parasites, fungi, and transformed (cancerous or pre-cancerous) cells.

The OpcA of the present invention may also be integrated, using recombinant techniques, into a live vaccine. Live vaccine vectors include: adenovirus, cytomegalovirus, and pox viruses such as vaccinia (U.S. Pat. No. 4,603,112,) and attenuated Salmonella strains (U.S. Pat. No. 4,550,081; Curtiss et al., 6 Vaccine 155-60 (1988)). In addition, OpcA epitopes may be incorporated into the flagella of attenuated bacterial strains. Similarly, inactivated recombinant viral vaccines may also be designed to include recombinant N. gonorrhoeae OpcA by known methods.

Vaccine preparations comprising N. gonorrhoeae OpcA include one or more adjuvants, such as surface active substances, e.g., hexadecylamine, octadecyl amino acid esters, octadecylamine, lysolecithin, dimethyl-dioctadecylammonium bromide, N,N-dicoctadecyl-N′,N′bis (2-hydroxyethyl-propane diamine), methoxyhexadecylglycerol, and pluronic polyols; polyamines, e.g., pyran, dextransulfate, poly IC, carbopol; peptides, e.g., muramyl dipeptide and derivatives thereof, dimethylglycine, tuftsin; oil emulsions; and mineral gels, e.g., aluminum hydroxide, aluminum phosphate, and lymphokines. Other suitable adjuvants include protein carriers such as tetanus toxoid.

Within the scope of the present embodiments are derivatives of the OpcA protein or peptides. “Derivative” is intended to include modifications of the native OpcA that retain the either the antigenicity or immunizing activity of the native polypeptides. The term is intended to include, without limitation, portions, fragments, or complexes of the protein, peptides, polypeptides, or fusion partner proteins made by recombinant DNA or other purification techniques whose amino acid sequences are identical or substantially identical (i.e., differ in a manner that does not substantially reduce the desired level of antigenicity) to that of the protein or that of an active portion thereof, or that lack or have different substituents (e.g., lack glycosylation or differ in glycosylation), and conjugates of the protein or such fragments, oligomers, polypeptides and fusion proteins and carrier proteins. The creation and use of such polypeptides and derivatives are well-known in the art.

It is also intended that the protein coding regions for use in the present invention could also be provided by altering existing opcA using standard molecular biological techniques that result in variants (agonists) of the peptides described herein. Such variants include, but are not limited to deletions, additions and substitutions in the amino acid sequence of the OpcA peptides, and are well-known in the art. For example, one class of substitutions is conserved amino acid substitutions. Such substitutions are those that substitute a given amino acid in the peptide by another amino acid of like characteristics. Typically seen as conservative substitutions are the replacements, one for another, among the aliphatic amino acids Ala, Val, Leu, and Ile; interchange of the hydroxyl residues Ser and Thr, exchange of the acidic residues Asp and Glu, substitution between the amide residues Asn and Gln, exchange of the basic residues Lys and Arg, replacements among the aromatic residues Phe, Tyr, and the like. Guidance concerning which amino acid changes are likely to be phenotypically silent is found in Bowie et al., 247 Science 1306-10 (1990).

Variant or agonist peptides may be fully functional or may lack function in one or more activities. Fully functional variants typically contain only conservative variations or variations in non-critical residues or in non-critical regions. Functional variants can also contain substitution of similar amino acids that result in no change or an insignificant change in function. Alternatively, such substitutions may positively or negatively affect function to some degree. Non-functional variants typically contain one or more non-conservative amino acid substitutions, deletions, insertions, inversions, or truncation or a substitution, insertion, inversion, or deletion in a critical residue or critical region.

Amino acids that are essential for function can be identified by methods known in the art, such as site-directed mutagenesis or alanine-scanning mutagenesis. Cunningham et al., 244 Science 1081-85 (1989). The latter procedure introduces single alanine mutations at every residue in the molecule. The resulting mutant molecules are then tested for biological activity such as epitope binding or in vitro ADCC activity. Sites that are critical for ligand-receptor binding can also be determined by structural analysis such as crystallography, nuclear magnetic resonance, or photoaffinity labeling. Smith et al., 224 J. MOL. BIOL. 899-904 (1992); de Vos et al., 255 Science 306-12 (1992).

Moreover, polypeptides often contain amino acids other than the twenty “naturally occurring” amino acids. Further, many amino acids, including the terminal amino acids, may be modified by natural processes, such as processing and other post-translational modifications, or by chemical modification techniques well known in the art. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as PROTEINS-STRUCTURE & MOLECULAR PROPERTIES (2nd ed., T. E. Creighton, W. H. Freeman & Co., New York, 1993). Many detailed reviews are available on this subject, such as by Wold, POSTTRANSLATIONAL COVALENT MODIFICATION OF PROILINS, 1-12 (Johnson, ed., Academic Press, New York, 1983); Seifter et al., 182 Meth. Enzymol. 626-46 (1990); and Rattan et al., 663 Ann. N.Y. Acad. Sci. 48-62 (1992). The secondary and tertiary structure of the peptides of the present invention may be determined by any number of techniques well-known in the art, or predicted by well-known methodologies such as Chou-Fasman secondary structure analysis. Accordingly, the peptides of the present invention also encompass derivatives or analogs in which a substituted amino acid residue is not one encoded by the genetic code, in which a substituent group is included (such as pegylation) as mentioned previously.

Similarly, the additions and substitutions in the amino acid sequence as well as variations, and modifications just described may be equally applicable to the amino acid sequence of anti-OpcA antibodies that may be raised against the proteins of the present invention by methodologies well known in the art, and are thus encompassed by the present invention.

The OpcA described herein may generate an immune response. The term “immune response” refers to a cytotoxic T-cell response and/or increased serum levels of antibodies specific to an antigen, or to the presence of neutralizing antibodies to an antigen. The immune response may indeed be sufficient to make the antigen of the invention useful as a vaccine for protecting human subjects from human pneumococcal infection. Additionally, antibodies generated by the antigen of the invention can be extracted and used to detect a bacterium in a body fluid sample. The term “protection” or “protective immunity” refers herein to the ability of the serum antibodies and/or cytotoxic T-cell response induced during immunization to protect (partially or totally) against a disease caused by an infectious agent, e.g., human N. gonorrhoeae. The use of the immunogenic polypeptides in a vaccine is expected to provide protective immunity to humans against severe gonococcal infection by, inter alia, inducing antibodies against gonococci.

The invention includes a method of providing an immune response and protective immunity to a patient against gonococcal-mediated diseases. The method includes administering an OpcA antigen of the invention to an animal or human. The OpcA antigen of the invention is preferably administered as a formulation comprising an effective amount of the antigen. A variety of physiologically acceptable carriers are known in the art, including for example, saline. Routes of administration, amounts, and frequency of administration are known to those skilled in the art for providing protective immunity to a recipient subject. Routes of administration include any method which confers protective immunity to the recipient, including, but not limited to, inhalation, intravenous, intramuscular, intraperitoneal, intradermal, mucosal, and subcutaneous. The antigen of the invention may be provided to a human subject by subcutaneous or intramuscular injection. A range of amounts and frequency of administration is acceptable so long as protective immunity of the recipient is achieved. For example, 5 μg to 20 μg can be administered by intramuscular injection between one and four times over a three month period.

Hence, the novel immunogenic OpcA polypeptides provided herein may be useful in a vaccine or in gonococcal vaccine development. For example, the polypeptide may be incorporated into a vaccine, either alone, as a component, or as a protein carrier for a polysaccharide conjugate vaccine. See, e.g., U.S. Pat. No. 5,866,135; No. 5,773,007; and No. 6,936,252.

Antibodies raised against the OpcA or portions of OpcA of the present invention are also encompassed herein. The ability of the anti-OpcA peptide antibodies to elicit cross-protection (protection against additional strains representing different gonococcal types, in which the intrinsic OpcA exhibits non-identical regions to those in the vaccine) are determined by further immunization and challenge experiments using additional challenge strains to verify broad cross-protection. Sera from immunized animals such as mice and rabbits are also tested for the ability to passively protect mice to determine if the protection is primarily through elicited antibody. Protection is further characterized in additional mouse models, and the potential that T-cells are involved in the protection is determined.

Additionally, the smallest portions of OpcA that are able to elicit protective antibodies may be identified in a mouse model of infection as described herein. This is done by making smaller recombinant portions, fragments, or polypeptides from the present immunogenic regions to determine the minimal effective epitopes.

The vaccine of the present invention may also comprise an anti-idiotypic monoclonal antibody or an immunogenic portion thereof. This approach is also known in the art (see U.S. patent application Publication No. 20060073139).

### EXAMPLES

### Example 1

### Strains of N. gonorrhoeae

Strains representing the diversity found in natural populations of N. gonorrhoeae were sequenced to identify a consensus sequence for the OpcA protein, including representative genotypes on the phylogenetic tree; representative isolates from different clinical diseases including disseminated gonococcal infection (DGI), inflammatory disease (PID) and paternal infections; and representative isolates from global geographic sources (North America, South America, Asia, Africa and Europe). A total of 210 gonococcal strains were examined. The reference strain FA1090 was obtained from Dr. Rice (Univ. Mass. Med. School, Worcester, Mass.). The reference strain MS11 purchased from The American Type Culture Collection (ATCC). Other strains were obtained from Dr. M. Bach (Cntr Biologics Evaluation & Res. FDA, Bethesda, Md.) and Dr. J. Zenilman (Johns Hopkins Univ. School Med., Baltimore, Md.). GC agar plates were prepared by using Difco GC medium base, bovine hemoglobin and IsoVitalex enrichment from Becton Dickinson (Franklin Lakes, N.J.) according to ATCC protocols available on-line. The bacterial strains were grown on GC agar plates at 37° C. in 5% CO₂ for 16 h. The bacterial cultures were used for preparation of outer-membrane protein, extraction of chromosomal DNA and bactericidal assay.

### Example 2

### PCR Amplification and DNA Sequencing of the opcA Gene

Two primers, P103 (5′-TTCGTTACCTCCGGCATCCG-3′) (SEQ ID NO:11) and P61 (5′-ACCATCAAATGAATATCCAT-3′) (SEQ ID NO:12), are used to amplify the opcA locus from N. gonorrhoeae strains. P103 is located at the glyA gene in the upstream region of opcA and P61 is located at dedA downstream. The PCR mixtures contain 1 μl of 10 mM deoxynucleoside triphosphates, 10 pmol of each primer, 0.1 μg of chromosomal DNA, 5 μl of 10×PCR buffer, and 1.5 U of Taq DNA polymerase (Perkin-Elmer), and sterile redistilled H₂O in a final volume of 50 μl. PCR amplification is performed using the following protocol: denaturation at 94° C. for 2 min, 30 cycles of amplification at 94° C. for 30 sec, 56° C. for 30 sec and 72° C. for 2 min, and a final extension at 72° C. for 4 min. The PCR products are analyzed by electrophoresis on a 1% agarose gel and stained with ethidium bromide. The PCR products are purified by QIAquick spin-column (Qiagen). DNA sequences are determined from both strands of three independent PCR products for each strain. DNA sequences are analyzed with the Genetics Computer Group package (GCG10.2-Unix, University of Wisconsin) and the Molecular Evolutionary Genetics Analysis software (MEGA2.1, Arizona State University).

### Example 3

### Cloning and Expression of Gonococcal opcA Gene

The sequence of the opcA gene from N. gonorrhoeae strain FA1090 (GenBank accession number AJ242836) is used to design two additional primers to amplify the entire opcA open reading frame and introduce BamHI and HindIII restriction sites for cloning. The amplified DNA fragments are cloned into the pRSETA expression vector (Invitrogen). E. coli JM109 (DE3) (Promega) is transformed with recombinant plasmids to express the proteins with N-terminal polyhistidine (6×His) tag for rapid purification with ProBond® resin. Expression of recombinant proteins is evaluated according to the appearance of bands in SDS-polyacrylamide gel electrophoreses. The recombinant OpcA protein is purified by affinity chromatography on a column of Xpress® Purification Kit (Invitrogen).

DNA manipulations. (i) DNA isolation. The bacterial genomic DNA was isolated from N. gonorrhoeae and E. coli using the QIAamp DNA Mini Kit (Qiagen, Valencia, Calif.). (ii) PCR amplifications. The PCR reaction mixtures contained 1 μl of 10 mM dNTPs, 10 μmol of each primer, 0.1 μg of chromosomal DNA, 5 μl of 10×PCR buffer and 1.5 U of Taq DNA polymerase (Promega), and sterile redistilled H₂O in a final volume of 50 μl. PCR amplification was performed on a PTC-200 thermocycler (MJ Research) using following protocol: denaturation at 94° C. for 2 min, 30 cycles of amplification at 94° C. for 30 sec, 56° C. for 30 sec and 72° C. for 2 min, and a final extension at 72° C. for 4 min. The PCR products were analyzed by electrophoresis on 1% agarose gel and stained with ethidium bromide. (iii) DNA sequencing. The PCR products from different gonococcal strains were purified by QIAquick spin-column (Qiagen). DNA sequences were determined from both strands of three independent PCR products for each strain as described (Zhu et al., 2002). The sequence traces were edited and assembled with ChromasPro (Version 1.33, Technelysium Pty). DNA sequences were analyzed with the Genetics Computer Group package (GCG10.2-Unix, University of Wisconsin) and the Molecular Evolutionary Genetics analysis software (MEGA 3.1, Arizona State University). (iv) Quantitative real-time PCR assays. The cell numbers of N. gonorrhoeae in adherence assays were determined by using quantitative real time PCR assays with primers and TaqMan probes specific to N. gonorrhoeae. The TaqMan probe was dual labeled with FAM fluorophore reporter at 5′-end and black hole quencher (BHQ-1) at 3′-end. A real-time PCR reaction mixture was prepared as described (Zhu et al., 2005) except that the master mix was TaqMan Universal PCR Master Mix (Applied Biosystems, Branchburg, N.J.) at the following conditions: denaturation and enzyme activation at 95° C. for 10 min and 40 cycles of 95° C. for 10 s and 60° C. for 1 min. PCR was performed on SmartCycler System (Cepheid, Sunnyvale, Calif.). A calibration curve was constructed using 10-fold serial dilutions of standard plasmid containing the target gene. The genomic copy numbers of gonococci were estimated on the basis of the threshold cycles and the calibration curve.

Cell lines and growth conditions. All cell culture media were supplemented with 10% inactivated fetal bovine serum and 2 mM L-glutamine. ME-180 (ATCC HTB33), an epithelial-like human cell line derived from cervical carcinoma, was obtained from ATCC. ME-180 was maintained in McCoy's 5A medium supplemented with 10% fetal calf serum and was grown in Dulbecco's modified Eagle medium (DMEM). All the cells were grown at 37° C. in a 5% CO₂ atmosphere.

SDS-PAGE and immunoblotting analysis. Protein samples were resolved by a gradient 4%-20% SDS-PAGE Precise Protein Gel (Pierce). The cell lysate and purified recombinant protein were suspended in SDS sample buffer (0.06 M Tris-HCl, pH 6.8, 10% glycerol [v/v], 2% SDS [w/v], 5% 2-βmercaptoethanol [v/v], 0.01% bromophenol blue [w/v]) and heated to 100° C. for 3 min, left at ambient temperature for 5 min before being loaded onto the gel at 10 μg per well. SDS-PAGE electrophoresis was performed with Tris-Hepes-SDS Running Buffer (Pierce) at 100 V for 1 h. After electrophoresis, the gel was prefixed with a 50% methanol and 7% acetic acid solution for 15 min, and then rinsed three times for 5 min with 200 ml of distilled water with gentle shaking. Separated proteins were stained with GelCode Blue Strain Reagent (Pierce). For immunoblotting analysis, separated proteins were transferred to polyvinylidene difluoride membrane (PVDF, Pierce) by Mini Trans-Blot Cell (Bio-Rad) at 100 V for 1 hr, and following incubation with blocking solution (1% BSA in PBST) for 30 min. The membrane was incubated with antisera diluted in blocking solution for 1 h, and immunological reactivity was detected using second antibody conjugated with alkaline phosphatase and 1-Step NBT/BCIP-Blotting substrate (Pierce).

### Example 4

### Systemic and Mucosal Immune Responses to Recombinant opcA Protein

The purified protein is used to immunize mice with adjuvant preparations that have the potential for use in humans. To present the protein in its native conformation for immunization, the OpcA protein is incorporated into liposomes prepared by dialysis-sonication (OpcA-liposomes). The recombinant protein is solubilized with the zwitterionic detergent Zwittergent 3-14 (OpcA-Zwit), as an alternative means of refolding denatured proteins into native conformations. To increase the immunogenicity of the recombinant protein, the immunomodulator MPLA is incorporated into OpcA-liposomes (Opc+MPLA−liposomes) and OpcA-Zwittergent micelles (OpcA+MPLA−Zwit). In addition, OpcA-liposome is mixed with liposomes incorporating MPLA (MPLA-liposomes). As a control, the protein is adsorbed to Al(OH)₃, the standard adjuvant routinely licensed for human use.

Female BALB/c mice at 6 to 7 weeks of age are used for immunizations, with blood and vaginal fluids samples taken before primary immunization. Four groups of forty mice of approximately equal weight are sequentially immunized with different protocols (group I, intranasally; II, intravaginally; III, intraperitoneally; IV, subcutaneously). Intranasal protocol is performed first. Individual mice within four groups are immunized with 20 μg of recombinant OpcA protein in each of the above preparations on days 0, 14, 28, and 46. Blood samples are taken on day 35 and the mice are terminally bled on day 60, and sera stored at −20° C. Vaginal washes are collected on days 35 and 60 by repeated flushing and aspiration of 30 μl of PBS containing 0.1% BSA (Sigma) and 1 mM phenylmethylsulfonyl fluoride (Sigma) as protease inhibitor. The vaginal washing will be repeated twice and the fluid specimens will be pooled and stored at −20° C.

### Example 5

### Immunological Analysis of Anti-opcA Antibodies

The systemic and mucosal immune response to the purified OpcA protein is studied initially by the reactivity of murine antisera and vaginal fluids in ELISA experiments. ELISA determines antibody titers of total IgA1, IgA2, IgM and IgG on microtiter plates coated with the recombinant OpcA protein. Immunoglobulin titers are expressed as the reciprocal of sample dilution that gave an A₄₉₀ value of 0.4 above the pre-immune sample. Antibodies raised against recombinant OpcA are also tested using homologous and heterologous strains in Western blotting analysis. The heterologous strains for Western blotting include all gonococcal OpcA variants found by sequencing opcA from 201 gonococcal strains and an additional 9 strains representing heterologous serovars (strain 880140, serotype IB14; 880250, IA02; 880288, IB03; 880447, IB01; 881051, IB05; 881096, IB07; 882602, IA06; 882916, IB04; 883021, IA05). Immunoblots are prepared on purified recombinant OpcA proteins, with 15% polyacrylamide gels and sample dilutions (sera, 1:200; vaginal fluids, 1:50). Immunoglobulin class and subclass-specific response to OpcA are tested. Direct binding assay, such as whole cell-ELISA, are performed after the positive antibodies are detected.

ELISA analysis of anti-OpcA antibody. Flat-bottom polystyrene 96-well plates (Nunc) were coated with 1 μg/ml recombinant OpcA protein in 0.05 M sodium carbonate buffer (pH 9.6) at 4° C. overnight. The plates were washed, and blocked with PBS containing 1% BSA, 0.05% Tween-20 at room temperature for 1 hr. 100 μL of serial dilutions of murine sera or vaginal wash fluid were incubated in the plates at 37° C. for 1 hr. After washing, antibody binding was detected by the second antibody conjugated with horseradish peroxidase and 1-Step™ Ultra TMB substrate (Pierce). The reaction was stopped after 15 mM by adding 50 μL of 2 M sulfuric acid, and the absorbance was measured at 450 nm with Multiskan MCC/340 microplate reader (Thermo Labsystems). To determine antibody subclass specific responses, a standard curve was generated by using Mouse Reference Serum and Mouse IgG-subclass Quantitation Kits (Bethyl Laboratories) according to the manufacturer instructions. Immunoglobulin concentrations in the samples were interpolated from the standard curve by using Ascent software (Thermo Labsystems).

### Example 6

### Biological Assays of Antibody Effect

Two assays are performed in assessment of the biological effect of anti-OpcA antibodies:

Bactericidal killing assay: Bactericidal assay was performed as described. McQuillen et al., 236 Enzymol. 137-47 (1994). Colonies of FA1090 were picked up from GC agar and suspended in 2 ml of MHB-II. The OD600 nm of bacterial suspension was adjusted with MHB-II to ≈0.20. This suspension contained about 10⁸ CFU/ml bacterial cells. The bacterial suspension was 1:100 diluted in MHB-II and then 1:100 further diluted in HBSS containing 1 mM MgC12 and 0.15 mM CaCl₂ (HBSS++) to a final concentration about 10⁴ CFU/ml bacterial cells. A bactericidal reaction mixture was prepared in 96-well microtiter plate by mixing 15 μl of diluted serum sample, 15 μl of HSC complement (10%), 25 μl of 10⁴ CFU/ml FA1090 bacterial suspension and 95 μl of HBSS++ to a final volume of 150 μl. The reaction mixture was incubated at 37° C. with shaking 40 rpm/min for 30 min. Twenty-five (25) μl of reaction mixture was sampled and plated on GC agar plate at two time points: T₀ₘᵢₙ (immediately) and T₃₀ₘᵢₙ (after 30 min incubation). Duplicate samples were collected and tested. The GC agar plates were incubated at 37° C., 5% CO₂ for 24 hr and all bacterial colonies on each plate were counted. The bactericidal activity was defined as serum dilution resulting 50% decrease in CFU per plate after 30 min of incubation compare to the CFU at time zero (T₃₀ₘᵢₙ/T₀ₘᵢₙ). Two antibodies were used as positive controls in this assay. One was a rabbit polyclonal antibody against whole gonococcal cells of strain ATCC 31426 (Abcam). Another was a monoclonal antibody 2C7 against gonococcal lipooligosaccharide LOS from Dr. Rice. Ngampasutadol et al., 102 P.N.A.S. USA 17142-47 (2005). Antibody #2C7 was purified through a protein-A affinity chromatography before use. Two negative controls were tested in each assay that included a reaction mixture with active HSC complement only and a reaction mixture with heat-inactivated HSC complement plus the sample serum or the control antibody.

Cell-adherence inhibition assay: The cell-adherence inhibition assay is performed using ME180 cells (human cervical cell line) from American Type Culture Collection. The ability of anti-OpcA antibodies to prevent adherence to epithelial cell by gonococci is determined in the presence or absence of the antibodies. Adherence inhibition is quantified by determining the ratio of cell-associated colony-forming units (cfu) to total cfu of the inoculum.

In case that anti-OpcA antibodies are not bactericidal, an opsonic phagocytosis assay is performed in assessment of opsonic effect of anti-OpcA antibodies. Opsonization of anti-OpcA antibodies will be measured. A reaction mixture of the opsonic phagocytosis assay consists of gonococcal strain FA1090 (1.25×10⁶/mL), human polymorphonuclear leucocytes (PMNL) (1.25×10⁶/mL), a titration of anti-OpcA antibodies diluted in Hanks balanced salts solution (HBSS), and 10% fresh human serum as a complement source. Duplicate reaction mixtures and control mixtures are prepared and tested in the assay. Viable bacteria are counted after the incubations at 37° C. Survival is expressed as the percentage of organisms at time zero that survived to 60 min.

### Example 7

### Construction of opcA-Knockout Mutant

To determine the role of opcA in the N. gonorrhoeae, the opcA knockout mutant is made by insertion of a kanamycin resistance cassette (Amersham Pharmacia Biotech) into the opcA gene in the N. gonorrhoeae chromosome. A kanamycin resistance cassette is chosen as a selective marker.

A fragment of 892 bp containing the whole coding region of opcA is amplified from strain FA1090 of N. gonorrhoeae by PCR. After purification through QIAquick spin-column (Qiagen), the PCR product is cloned into pCR® T7/NT-TOPO plasmid (Invitrogen). The opcA insert in the recombinant plasmid is confirmed by nucleotide sequencing.

The recombinant plasmid is digested with restriction endonuclease SphI that cuts the opcA gene at position 213 after ATG start codon. The overhangs at the end of DNA fragment is filled in with T4 DNA polymerase (Promega), purified through QIAquick spin-column, and then added to HincII-digested kanamycin resistance cassette from pUC4Kan (Amersham Pharmacia). The blunt ends is ligated using T4 DNA ligase (Promega).

After transformation into E. coli TOPO10F′ cells and selection on kanamycin-containing plates, colonies will be examined by PCR for the constructs with the kanamycin cassette in the same transcriptional orientation as the opcA gene. The plasmid construct is linearized by digestion with EcoR I, and will be used to transform strains FA1090 and F62 of N. gonorrhoeae by electroporation according to the method of A. Elizabeth [ ]. Cells will be plated upon BHI-kanamycin; then transformants are confirmed as opcA mutants by PCR and by Southern blot hybridization.

### Example 8

### Site-Directed Mutations of opcA Mutant

If deletion of whole opcA is lethal to N. gonorrhoeae, additional experiments are performed to create site-directed mutations at the opcA regions coding for surface loops of OpcA. The opcA gene is cloned into pALTER®-1 vector (Promega Corp. Madison, Wis.). The gene regions corresponding to each surface loop of the OpcA protein are used as targets for mutagenesis analysis. The mutangenic oligonucleotides are designed and synthesized. The site-directed mutagenesis of gonococcal opcA gene is done by using the commercial available kit of in vitro mutagenesis system (Promega). The mutations are confirmed by DNA sequencing. Strain FA1090 of N. gonorrhoeae is transformed with various constructs to test whether the mutagenesis at the surface loops of the opcA gene affect the ability of gonococcal adhesion and invasion.

### Example 9

### Adherence Assays

Bacterial invasion of human mucosal cells is considered to be a primary event in the pathogenesis of a gonococcal infection. Whether the biological functions of N. meningitidis OpcA, adhesion to proteoglycan and vitronectin are preserved in N. gonorrhoeae OpcA is determined. Gonococcal binding activity to primary cultures of human corneal epithelial cells is compared between opcA-knockout mutant and wild type strain as follows:

Primary cultures of human cervical cell lines may be used in adherence assays. Cervical cells are grown on 12-mm circular glass or thermanox coverslips in 1 ml of medium. Before the start of the infection, the medium is replaced with 1 ml of DMEM supplemented with 5% FCS. Gonococci are grown on GC agar plates (14 h, 5% CO₂), suspended in tissue culture medium, and added to the cells (2×10⁷ per well). After 1 hr incubation (37° C., 10% CO₂), the infection is stopped by rinsing the cells three times with 1 ml of Dulbecco's PBS (DPBS) to remove unbound bacteria, followed by fixation (at least 30 min, room temperature) in 0.1% glutaraldehyde/1% paraformaldehyde in DPBS. Specimens are stained with crystal violet (0.007% in distilled water). Infected cells are photographed under microscope, and photoprints are counted directly to determine the number of adherent bacteria per cell.

In cases where large numbers of adherent bacteria and microcolony formation preclude accurate counting, quantitation of adherence is confirmed as follows: after 1 hr incubation, nonadherent bacteria are removed by washing five times with assay media. The monolayers and cell-associated bacteria are then recovered by treatment with 0.25% trypsin for 5 min at 37° C. The recovered bacteria are plated on agar after dilution, and relative adherence is quantified by determining the ratio of cell-associated colony-forming units (cfu) to total cfu of the inoculum.

Based on the biological materials of recombinant OpcA and specific antibodies obtained the purity, safety, and efficiency of OpcA-based vaccines is evaluated. This also demonstrates whether anti-OpcA antibodies can confer protective immunity to N. gonorrhoeae in the animal model. Monoclonal antibodies are generated using the recombinant OpcA protein. The specific epitopes are mapped on the protein sequences and structure. Both recombinant OpcA protein vaccine and conjugated-peptide vaccine are used for immunization. The efficiency of the vaccine is measured by groups of mice by immunization following the bacterial challenge. The purity and safety of the vaccines are confirmed by standard tests described in the European Pharmacopoeia and the U.S. Code of Federal Regulations.

### Example 10

### Preparation to Monoclonal Antibody Specific to opcA

In order to identify the protective epitopes on gonococcal OpcA protein, a panel of monoclonal antibodies is prepared. The recombinant OpcA protein may be used as an antigen for the production of hybridomas. Briefly, four 6-week-old BALB/c female mice are immunized subcutaneously with 20 μg of OpcA protein in 0.1 ml of saline mixed with 0.1 ml of Freund's complete adjuvant (Difco), followed by booster injection of OpcA protein on day fourteen with the same mixture. Blood is taken from each mouse and the antibody response is measured by ELISA. The mouse with the highest serum antibody titer is selected as the spleen donor and is given a booster injection of 20 μg of OpcA protein in saline three days prior to fusion. Serum samples are collected from non-immunized/immunized mice and serve as negative/positive controls, respectively.

SP2/0-Ag14 murine myeloma cells are grown in Dulbecco's modified Eagle medium (DMEM; Invitrogen) supplemented with 10% heat-inactivated bovine fetal serum, 100 U gentamicin/ml and 2 mM L-glutamine (Invitrogen). The fusion of spleen cells from the selected mouse with SP2/0-Ag myeloma cells is performed by using a standard method (e.g., Köhler & Milstein) with 50% (w/v) polyethylene glycol (molecular mass, 3000-3700 Da; Sigma). The fused cells are cultured in five 96-well microtitre plates in the presence of hypoxanthine, aminopterin and thymidine (HAT; Sigma), and incubated at 37° C. in a humid atmosphere of 5% CO₂. Hybridoma culture supernatants are examined for the presence of antibodies by ELISA. Hybridoma cells producing antibodies are cloned twice by limiting dilution with Hybridoma Enhancing Supplement (Sigma).

Hybridoma culture supernatants are screened for antibodies by ELISA using recombinant OpcA protein as an antigen. A 96-well microtitre plate (Nunc) is coated with 2 μg of OpcA protein per well in carbonate buffer (pH 9.6) and incubated at 4° C. overnight. The plate is washed three times with PBS containing 0.05% Tween-20 (PBST). The plate will be blocked with 100 μl PBS containing 1% BSA at 37° C. for 1 hr and washed with PBST three times. Then, 100 μl of hybridoma culture supernatants, or the positive and negative controls (sera from immunized and non-immunized mice), is added each well. After three washes with PBST, 100 μl of goat anti-mouse IgG conjugated to horseradish peroxidase (Pierce, diluted in PBST per manufacturer's instructions) is added to each well. The plate is incubated at 37° C. for 1 hr and washed with PBST four times. A 50 μl aliquot of One-Step™ Ultra TMB substrate (Pierce) is added and incubated at room temperature for 15 min and the reaction is stopped by adding 50 μl of 2 M sulfuric acid. The absorbance is measured at 450 nm with Multiskan MCC/340 microplate reader (Thermo Labsystems). Hybridomas showing at least 30% OD value of the positive control are considered positive and selected for further characterization. The isotypes of mAbs are determined by an ELISA with a mouse monoclonal subisotyping kit containing rabbit anti-mouse IgG1, IgG2a, IgG2b, IgG3, IgM and IgA (Bio-Rad).

Murine mAbs against recombinant OpcA protein in the culture supernatant or ascites fluid are purified by using a Sephadex G-25 precolumn (to remove phenol red from the medium) and anion exchange column chromatography (Resource 15Q anion). The concentration of pure antibody is quantified by absorbance at 280 nm and by a standard BCA protein assay (Pierce). The purity of the antibody is confirmed by sodium dodecyl sulfate-polyacrylamide gel electrophoresis. The antigenic specificity is determined by OpcA-ELISA.

### Example 11

### Identification and Characterization of Protective Epitope(S) on the opcA Protein

To determine subunit reactivity and to discriminate between antibodies recognizing linear and discontinuous epitopes, all mAbs are screened for the ability to detect OpcA protein by Western blotting. The Phase I protocols for gel electrophoresis and immunoblotting analysis will be used in Phase II study. The OpcA protein will be separated by gel electrophoresis under nondenaturing and reducing-denaturing conditions, respectively. Based on the reactivity on immunoblotting, we will determine if the mAbs are directed at conformational or linear epitopes.

Mapping of epitopes on the OpcA protein using peptides scanning. To further define the epitopes recognized by the panel of mAbs, a series of overlapping 20-mer peptides covering all surface loops of the OpcA protein is synthesized based on the OpcA sequence. Each mAb is tested for binding of the loop peptide using a peptide-ELISA. Lyophilized peptides are solubilized in 25 mM ammonium sulfate, pH 4.5, diluted to a concentration of 10 μg/ml in PBS and 50 μl/well are coated to Nunc-immuno maxisorb microtiter plates (Nalge Nunc, Rochester, N.Y.) at 4° C. overnight. Plates are washed three times with PBS supplemented with 0.05% Tween-20 (PBST). The plate is blocked with 100 μl of PBS containing 1% BSA at room temperature for 1 hr. 100 μl of the mAb solutions are incubated in the plates at 37° C. for 1 hr. After washing, antibody binding is detected by a second antibody, conjugated with horseradish peroxidase, and 1-Step™Ultra TMB substrate (Pierce). The reaction is stopped after 15 min by adding 50 μl of 2 M sulfuric acid. The absorbance is measured at 450 nm with Multiskan MCC/340 microplate reader (Thermo Labsystems, Franklin, Mass.).

The bactericidal activity of the MAbs is useful to determine the epitopes of the gonococcal OpcA protein, which are potentially protective. One of the important strengths of this study involves the utilization of monoclonal antibodies in the bactericidal assay. The mouse polyclonal antisera against gonococcal OpcA protein had bactericidal activity, as shown herein, yet the possibility of undetected contaminating antibodies to other bacterial proteins might not be excluded. This is not the case when the OpcA-specific monoclonal antibodies are used in bactericidal assays. The bactericidal activity of anti-OpcA antibody is further confirmed by using monoclonal antibody. Two formats of bactericidal assay include direct bactericidal assay in which monoclonal antibodies are directly used in the bactericidal assays; and an inhibitory bactericidal assay, in which monoclonal antibodies are used to block the binding sites to validate bactericidal activity of the serum anti-OpcA antibody. In addition, bactericidal activity of each monoclonal antibody will be tested against the loop-deletion mutants and the wild-type stains. The wild-type strain serves as positive control, whereas loop-deletion mutant as negative control. This study identifies the potential epitopes defined by monoclonal antibody that elicits biological protective activity.

Conservation of the OpcA epitope may also be recognized by mAbs. To investigate the conservation of the OpcA epitope associated with protective activity, various strains of N. gonorrhoeae are analyzed by Western blot assay with the defined anti-OpcA monoclonal antibodies. The strains with different epidemiological background will be provided by Dr. Jonathan Zenilman of the Johns Hopkins Medical Center (Baltimore, Md.). The reference strain FA1090 is used as control. The reactivity of these strains to the defined monoclonal antibody is compared. The conserved epitope in all strains will be identified.

### Example 12

### Identification of opcA Domains Implicated in Bacterial Adherence

In order to provide further evidence that the protective epitopes are exposed on the surface of the membrane, four isogenic OpcA loop-deficient mutants that lack the predicated loops 1, 2, 3 and 4 are constructed. The prediction of the structure of gonococcal OpcA protein by a homologous topology model showed that the OpcA protein is a membrane-spanning protein with five surface-exposed loops (Prince et al., 2002; Zhu et al., 2003). Loop 5 has the shortest length due to deletion of four amino acids, therefore it will not be included in this deletion study. Four loop-deletion mutants constructed in this project can be used both for confirmation of surface locations for protective epitopes and for identification of OpcA domains important in bacterial adherence. Four plasmids with an opcA gene containing the loop-deletions are constructed in E. coli. The linearized constructs will be transformed into gonococcal genome through homologous recombination. The loop-deletion mutants will be confirmed by PCR and DNA sequencing. By comparison with wild-type strain, these loop-deletion mutants will serve as controls to identify the important domains of the OpcA protein for bacterial adherence and bactericidal activity.

Plasmid pOPC-R11 described above carries the complete opcA gene from strain FA1090. This plasmid is used for the construction of a delivery plasmid by insertion of kanamycin resistance cassette, downstream of the opcA gene. The pOPC-R11 plasmid is digested with restriction endonuclease HindIII, which cuts the site immediately after TAA stop codon. The overhangs at the end of DNA fragment is filled with T4 DNA polymerase (Promega, Madison, Wis.), purified through QIAquick spin-column (Qiagen, Germantown, Md.), and added to HincII-digested kanamycin resistance cassette (Kan) from pUC4Kan (GE Healthcare, Piscataway, N.J.). The blunt ends are ligated using T4 DNA ligase (Promega). After transformation into E. coli TOP10 cells and selection on LB agar plates containing 50 μg/ml kanamycin, colonies are examined by PCR for constructs with the kanamycin cassette in the same transcriptional orientation as the opcA gene. The plasmid containing the expected insertion of Kan is purified by using Qiagen Spin Miniprep Kit (Qiagen).

The opcA gene from strain FA1090 is used for construction of the loop deletion by a two-step PCR protocol. Ho et al., 77 Gene. 51-59 (1989) Horton et al., 77 Gene. 61-68 (1989). In the first step, two fragments are synthesized by PCR with an overlap, where the sequences encoding the target loop is deleted. One fragment is generated with primers A and B. Primer A is the same primer O13 used above, which contains the BamHI site at the 5′-end. Primer B has two regions, which anneal to the flanking regions of the deletions, respectively. Another fragment is generated with primer C in conjunction with primer D. Primer D is the same primer O10 used above, which contains the HindIII restriction site at the 5′-end. Primer C is the complementary strand of primer B, which allows two PCR fragments overlapped with an internal deletion. These two fragments are then used together in a second reaction along with primers A and D to generate the opcA fragment with a loop deletion. Primers B and C are specific to each target loop, which will be designed in Phase II study. The resulting amplicon from the second PCR is a fusion fragment consisting of a loop deletion that can be used to replace the corresponding delivery plasmid. The PCR amplicon is digested with BamHI and HindIII and exchanged with the analogous fragment, derived from the plasmid expressing wild-type OpcA protein. DNA sequencing is performed to confirm that the deletions are constructed correctly.

The recombinant plasmid containing opcA-loop deletions are transformed into strain FA1090 to replace the wild-type of the opcA gene. The loop-deletion plasmid is linearized by digestion with BamHI, and is used to transform strain FA1090 of N. gonorrhoeae by electroporation. Cells are plated upon GC agar plate containing 50 μg/ml kanamycin. Transformants are confirmed as opcA loop-deletion mutants by PCR and by Southern blot hybridization.

The loop-deletion mutants derived from reference strain FA1090 are used to investigate the contributions of the important domains in the bactericidal immune responses and the interactions between N. gonorrhoeae, and host cells. In the adherence assay, human epithelial ME-180 cell line is cultured in 24-well plate for bacterial infection. Adherence of the mutant containing each loop-deletion to ME-180 will be compared with that of the FA1090 wild-type strain. The difference in bacterial adherence is examined by using Immunofluorescence staining and quantitative real-time PCR. The results allow identification of the domains of gonococcal OpcA protein implicated in bacteria adhesion.

### Example 13

### Effect of opcA Immunization on Gonococcal Vaginal Infections in Estradiol-Treated Mouse Model

N. gonorrhoeae causes natural infection only in humans. A major problem in understanding the diseases caused by the gonococcus is the lack of animal models using gonococcal infections. Development of animal models with N. gonorrhoeae genital tract infection has been unsuccessful in a number of hosts, including baboons, pig-tailed macaques, monkeys (rhesus, squirrel, owl, and capuchin), marmosets, rabbits, and guinea pigs. Arko, 2 Clin Microbiol Rev. S56-59 (1989). It was found that estradiol treatment prolongs vaginal colonization of N. gonorrhoeae in germ-free female mice. Taylor-Robinson et al., 9(5) Microb. Pathog. 369-73 (1990). An estradiol-treated murine model of gonococcal infection was subsequently developed by Dr. Ann Jerse of Uniformed Services University of the Health Sciences. Jerse, 67 Infect Immun. 5699-708 (1999).

This murine model has been used for assessing vaccine potential and determining biological function of several gonococcal components, including Opa, TbpB, PorB, OmpA, sialyltransferase, lactate permease, catalase, and multiple transferable resistance efflux complexes. See Jerse, 1999; Jerse et al., 70 Infect Immun. 2549-58 (2002); Ngampasutadol et al., 2005; Simms & Jerse, 74 Infect Immun. 2965-74 (2006); Wu & Jerse, 74 Infect. Immun. 4094-103 (2006); Exley et al., 75 Infect Immun. 1318-24 (2007); Soler-García & Jerse, 75 Infect Immun. 2225-33 (2007); Serino et al., 64 Mol. Microbiol. 1391-1403 (2007); Warner et al., 196 J. Infect. Dis. 1804-12 (2007).

Estradiol-treated BALB/c mice are used for assessing the vaccine potential of gonococcal OpcA protein, specifically evaluating (i) Capability of OpcA-induced immune response on the prevention gonococcal vaginal infections; (ii) Effect on the inactivation of the opcA gene on gonococcal vaginal colonization and duration; and (iii) Effect of both in vitro and in vivo conditions on the expression of the gonococcal opcA gene. The gonococcal OpcA protein serves as a virulence factor, and a protective antigen against gonorrhea. The pathogenesis study uses competitive infections with the wild type and opcA mutant strains in the 17-β-estradiol-treated mouse model to determine if the mutant has a survival or colonization defect in vivo. For immunization/challenge studies, the protective efficacy of recombinant OpcA (rOpcA) as a vaccine antigen delivered in two immunization schemes were tested, and were shown to elicit a high titer OpcA-specific antibody in mice.

To test whether OpcA plays a role in gonococcal genital tract infection, a group of five 17-β-estradiol-treated BALB/c mice are inoculated with a mixed bacterial suspension that is composed of similar numbers of wild type and opcA mutant gonococci. Vaginal mucus is cultured every-other-day for seven days and the relative number of wild type and mutant colony forming units (CFU) recovered (output) will be compared to that of the inoculum (input). The competitive index (CI) is calculated at each time point [CI=mutant CFU/wild type CFU (output)] divided by mutant CFU/wild type CFU (input), and a CI≦0.2 will be considered evidence of attenuation. Results are confirmed by fluorescent antibody staining of vaginal smears from infected mice using rabbit anti-Gc antisera (total # gonococci) and OpcA-specific antibody (wild type gonococci), with secondary antibodies conjugated to various dyes. The number of wild type and opcA mutant gonococci seen per field and associated with murine epithelial cells will be compared. Should the mutant show evidence of attenuation in vivo, the experiment is repeated with a group of mice inoculated with wild type gonococci mixed with a complemented version of the opcA mutant. A construct complemented mutant using pGCC chromosomal integration vectors allows expression of a wild type gene from an ectopic site. Complementation is confirmed by PCR and western blot with OpcAspecific antiserum.

To test the efficacy of rOpcA in preventing experimental murine infection, four groups of BALB/c mice including two test groups and two adjuvant control groups, (n=15 mice each) are immunized using the same protocol as described above. Data obtained herein showed that bactericidal activity of anti-OpcA was, significantly, induced in two immunization groups. In this Example, the test groups are immunized respectively with (i) The rOpcA solution (2 mg/ml) mixed with an equal volume of 1 mg/ml cholera toxin subunit B (Sigma); (ii) Protein-Zwittergent mixture containing 1 mg/ml recombinant rOpcA protein, 8 mg/ml of Zwittergent 3-14 (N-tetradecyl-N,N-dimethyl-3-ammonio-1-propanesulfonate, Sigma) and 0.2% SDS (w/v). The adjuvant control groups will be given the adjuvant alone, via the same route (n=2×15 mice). Serum and vaginal washes will be collected two weeks after the second immunization and analyzed for OpcA-specific titers. Three weeks after the final (third) immunization, all mice are given 17-βestradiol and treated with antibiotics, as per infection protocol. Two days after the final immunization, all mice are challenged intravaginally with N. gonorrhoeae. Vaginal mucus is cultured every other day for twelve days and the average number of CFU recovered over time will be compared by ANOVA. The average duration of colonization will be analyzed using the Log Rank test.

### Example 14

### OpcA Immunization of Gonococcal Vaginal Infection in Transgenic Mouse Model

Development of human C4BP transgenic mouse model has shown that gonococci display an array of surface antigens. These antigens influence the binding and function of human complement regulatory proteins, enabling the organism to escape immune surveillance and act as a uniquely human pathogen (Ngampasutadol et al., 2005; Ngampasutadol et al., 24 Vaccine 157-70 (2006); Ngampasutadol et al., 180 J. Immunol. 3426-35 (2008); Madico et al., 178. J. Immunol. 4489-97 (2007); Ram et al., 75 Infect. Immun. 4071-81 (2007). Non-human mammalian complement regulators (factor H and C4b binding protein (C4BP) being the major ‘upstream’ complement regulators) do not bind to gonococci (Ngampasutadol et al., 2005, 2006, 2008). Transgenic mice that express human factor H and human C4b binding protein (C4BP) are developed, as are mice that express the human form of the complement regulators. This is done by introducing the genes for the human complement regulators into the mouse eggs after the eggs have been fertilized by male sperm. The eggs are then placed into the womb of the female mouse where they grow into embryos. When new mice (called transgenic mice) are born, some of them have “inherited” the human gene for the complement regulators. These mice are then used to test whether the gonococcal organism survives best in mice with or without the human genes. Prolonged survival of the gonococcus in the transgenic mice suggests that the mice are expressing the human form of the complement regulator, or regulators, and are susceptible to gonococcal infections. These animals are then used to test whether vaccines can prevent gonococcal infection.

To assess protective immunity of OpcA-based vaccine, HC4BP transgenic mice are used in the second immunization/challenge experiment of Phase II. After intranasal immunization with the OpcA protein to C4BP transgenic mice, gonococcal strain FA1090 is used in the challenge experiment. Because gonococcal bacteria can use outer membrane porin molecules to bind the classical pathway of complement down-regulatory protein C4BP, they may evade the killing of the mouse complement system. By using C4BP transgenic mice, FA1090 are more resistant to the vaccine at the HC4BP background, making the challenge experiment stricter in testing vaccine efficiency. The immunization/challenge experiments using C4BP transgenic mice is performed in which two groups of HC4BP transgenic mice are immunized with gonococcal OpcA protein using the same protocol as that for estradiol-treated mice. The test results obtained from both animal models are compared.

The claimed invention is:

1. An immunogenic composition comprising a recombinant N. gonorrhoeae OpcA or a portion of fragment thereof.

2. The immunogenic composition of claim 1 further comprising an adjuvant.

3. The immunogenic composition of claim 2, wherein said adjuvant is selected from the group consisting of alum, cholera toxin B subunit, liposome, micelle, and R-700.

4. The immunogenic composition of claim 1 or 2, further comprising an antigen of N. gonorrhoeae other than OpcA.

5. The immunogenic composition of claim 4, wherein the antigen of N. gonorrhoeae is selected from the group consisting of Pili, Opa, Rmp, Tbp, PorB, and LOS components of N. gonorrhoeae, and combinations thereof.

6. The immunogenic composition of claim 1 or 2, further comprising an antigen of H. influenzae, N. meningitides, N. Polysaccharea, or S. pneumoniae.

7. The immunogenic composition of claim 1 or 2, further comprising a hapten conjugated to said recombinant N. gonorrhoeae OpcA.

8. The immunogenic composition of claim 1, wherein said recombinant N. gonorrhoeae OpcA is expressed in a live recombinant vector.

9. The immunogenic composition of claim 8, wherein said vector is selected from the group consisting of adenovirus, cytomegalovirus, pox virus, and attenuated Salmonella strains.

10. A method of eliciting an immune response in a subject by administering to the subject a composition comprising N. gonorrhoeae OpcA or a portion or fragment thereof.

11. The method of claim 10, wherein said composition further comprises one or more adjuvant.

12. The method of claim 11, wherein said adjuvant is selected from the group consisting of alum, cholera toxin B subunit, liposome, micelle, and R-700.

13. The method of claim 11, wherein said administering is selected from the group consisting of inhalation, intravenous, intramuscular, intraperitoneal, intradermal, intranasal, mucosal, and subcutaneous administration.

14. The method of claim 11, wherein said composition is delivered to the subject through mucosal administration.

15. A method of diagnosing an infection due to N. gonorrhoeae, said method comprising contacting a biological sample with a recombinant N. gonorrhoeae OpcA or a portion of fragment thereof, under conditions whereby an antibody/antigen complex is formed if antibodies to the recombinant N. gonorrhoeae OpcA are present in the biological sample, and detecting said complex.

16. A kit for diagnosing N. gonorrhoeae infection comprising a recombinant N. gonorrhoeae OpcA or a portion of fragment thereof.
